SEQUENCE LISTING (30 sequences)

SEQ ID NO: 1 - DNA, 2058 bp, Homo Sapiens
  CDS (1)..(2058)
atgctgctgctgcccctgctgctgcccgtgctgggggcggggtccctgaacaaggatcccagttacagtcttcaagtgcagaggcaggtgccggtgccggagggcctgtgtgtcatcgtgtcttgcaacctctcctacccccgggatggctgggacgagtctactgctgcttatggctactggttcaaaggacggaccagcccaaagacgggtgctcctgtggccactaacaaccagagtcgagaggtggcaatgagcacccgggaccgattccagctcactggggatcccggcaaagggagctgctccttggtgatcagagacgcgcagagggaggatgaggcatggtacttctttcgggtggagagaggaagccgtgtgagacatagtttcctgagcaatgcgttctttctaaaagtaacagccctgactaagaagcctgatgtctacatccccgagaccctggagcccgggcagccggtgacggtcatctgtgtgtttaactgggctttcaagaaatgtccagccccttctttctcctggacgggggctgccctctcccctagaagaaccagaccaagcacctcccacttctcagtgctcagcttcacgcccagcccccaggaccacgacaccgacctcacctgccatgtggacttctccagaaagggtgtgagcgcacagaggaccgtccgactccgtgtggcctatgcccccaaagaccttattatcagcatttcacatgacaacacgtcagccctggaactccagggaaacgtcatatatctggaagttcagaaaggccagttcctgcggctcctctgtgctgctgacagccagccccctgccacgctgagctgggtcctgcaggacagagtcctctcctcgtcccacccctggggccccagaaccctggggctggagctgcgtggggtaagggccggggattcagggcgctacacctgccgagcggagaacaggcttggctcccagcagcaagccctggacctctctgtgcagtatcctccagagaacctgagagtgatggtttcccaagcaaacaggacagtcctggaaaacctcgggaacggcacatccctcccggtcctggagggccaaagcctgcgcctggtctgtgtcacccacagcagccccccagccaggctgagctggacccggtggggacagaccgtgggcccctcccagccctcagaccccggggtcctggagctgccacccattcaaatggagcacgaaggagagttcacctgccacgctcagcaccctctgggctcccagcacgtctctctcagcctctccgtgcactaccctccacagctgctgggcccctcctgctcctgggaggctgagggtctgcactgcagctgctcctcccaggccagcccggccccctctctgcgctggtggcttggggaggagctgctggaggggaacagcagtcagggctccttcgaggtcacccccagctcagccgggccctgggccaacagctccctgagcctccatggagggctcagctccggcctcaggctccgctgtaaggcctggaacgtccacggggcccagagtggctctgtcttccagctgctaccagggaagctggagcatgggggaggacttggcctgggggctgccctgggagctggcgtcgctgccctgctcgctttctgttcctgccttgtcgtcttcagggtgaagatctgcaggaaggaagctcgcaagagggcagcagctgagcaggacgtgccctccaccctgggacccatctcccagggtcaccagcatgaatgctcggcaggcagctcccaagaccacccgcccccaggtgcagccacctacaccccggggaagggggaagagcaggagctccactatgcctccctcagcttccagggcctgaggctctgggagcctgcggaccaggaggcccccagcaccaccgagtactcagagatcaagatccacacaggacagcccctgaggggcccaggctttgggcttcaattggagagggagatgtcagggatggttccaaag

SEQ ID NO: 2 - protein, 686 aa, Homo Sapiens
MLLLPLLLPVLGAGSLNKDPSYSLQVQRQVPVPEGLCVIVSCNLSYPRDGWDESTAAYGYWFKGRTSPKTGAPVATNNQSREVAMSTRDRFQLTGDPGKGSCSLVIRDAQREDEAWYFFRVERGSRVRHSFLSNAFFLKVTALTKKPDVYIPETLEPGQPVTVICVFNWAFKKCPAPSFSWTGAALSPRRTRPSTSHFSVLSFTPSPQDHDTDLTCHVDFSRKGVSAQRTVRLRVAYAPKDLIISISHDNTSALELQGNVIYLEVQKGQFLRLLCAADSQPPATLSWVLQDRVLSSSHPWGPRTLGLELRGVRAGDSGRYTCRAENRLGSQQQALDLSVQYPPENLRVMVSQANRTVLENLGNGTSLPVLEGQSLRLVCVTHSSPPARLSWTRWGQTVGPSQPSDPGVLELPPIQMEHEGEFTCHAQHPLGSQHVSLSLSVHYPPQLLGPSCSWEAEGLHCSCSSQASPAPSLRWWLGEELLEGNSSQGSFEVTPSSAGPWANSSLSLHGGLSSGLRLRCKAWNVHGAQSGSVFQLLPGKLEHGGGLGLGAALGAGVAALLAFCSCLVVFRVKICRKEARKRAAAEQDVPSTLGPISQGHQHECSAGSSQDHPPPGAATYTPGKGEEQELHYASLSFQGLRLWEPADQEAPSTTEYSEIKIHTGQPLRGPGFGLQLEREMSGMVPK

SEQ ID NO: 3 - protein, 6 aa, Artificial Sequence
  conserved sequence
LHYASL

SEQ ID NO: 4 - protein, 6 aa, Artificial Sequence
  conserved sequence
TEYSEI

SEQ ID NO: 5 - protein, 8 aa, Artificial Sequence
  Flag Peptide
DYKDDDDK

SEQ ID NO: 6 - protein, 5 aa, Artificial Sequence
  Peptide Linker sequence
GGGGS

SEQ ID NO: 7 - protein, 6 aa, Artificial Sequence
  Peptide Linker sequence
GGGGSX

SEQ ID NO: 8 - protein, 12 aa, Artificial Sequence
  Peptide Linker sequence
GKSSGSGSESKS

SEQ ID NO: 9 - protein, 14 aa, Artificial Sequence
  Peptide Linker sequence
GSTSGSGKSSEGKG

SEQ ID NO: 10 - protein, 18 aa, Artificial Sequence
  Peptide Linker sequence
GSTSGSGKSSEGSGSTKG

SEQ ID NO: 11 - protein, 18 aa, Artificial Sequence
  Peptide Linker sequence
GSTSGSGKPGSGEGSTKG

SEQ ID NO: 12 - protein, 14 aa, Artificial Sequence
  Peptide Linker sequence
EGKSSGSGSESKEF

SEQ ID NO: 13 - protein, 27 aa, Artificial Sequence
  Leucine Zipper sequence
PDVASLRQQVEALQGQVQHLQAAFSQY

SEQ ID NO: 14 - protein, 33 aa, Artificial Sequence
  Leucine Zipper sequence
RMKQIEDKIEEILSKIYHIENEIARIKKLIGER

SEQ ID NO: 15 - protein, 5 aa, Artificial Sequence
  Localization sequence
KKKRK

SEQ ID NO: 16 - protein, 26 aa, Artificial Sequence
  Localization sequence
MLRTSSLFTRRVQPSLFRNILRLQST

SEQ ID NO: 17 - protein, 4 aa, Artificial Sequence
  Localization sequence
KDEL

SEQ ID NO: 18 - protein, 4 aa, Artificial Sequence
  Localization sequence
CAAX

SEQ ID NO: 19 - protein, 4 aa, Artificial Sequence
  Localization sequence
CCXX

SEQ ID NO: 20 - DNA, 26 bp, Artificial Sequence
  Forward Primer
cagcctctccgtgcactaccctccac

SEQ ID NO: 21 - DNA, 32 bp, Artificial Sequence
  Reverse Primer
gacctcttcactttggaaccatccctgacatc

SEQ ID NO: 22 - protein, 364 aa, Homo sapiens
MPLLLLLPLLWAGALAMDPNFWLQVQESVTVQEGLCVLVPCTFFHPIPYYDKNSPVHGYWFREGAIISGDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 23 - protein, 551 aa, Homo sapiens [sequence omitted]

SEQ ID NO: 24 - protein, 442 aa, Homo sapiens
MLPLLLPLLWAGALAQERRFQLEGPESLTVQEGLCVLVPCRLPTTLPASYYGYGYWFLEGADVPVATNDPDEEVQEETRGRFHLLWDPRRKNCSLSIRDARRRDNAAYFFRLKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGVLGAVWGASITTLVFLCVCFIFRVKTRRKKAAQPVQNTDDVNPVMVSGSRGHQHQFQTGIVSDHPAEAGPISEDEQELHYAVLHFHKVQPQEPKVTDTEYSEIKIHK

SEQ ID NO: 25 - protein, 467 aa, Homo sapiens
MLLLLLLPLLWGRERVEGQKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPPHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVGGAGATALVFLSFCVIFIVVRSCRKKSARPAADVGDVGMKDANTIRGSASQGNLTESWADDNPRHHGLAAHSSGEEREIQYAPLSFHKGEPQDLSGQEATNNEYSEIKIPK

SEQ ID NO: 26 - protein, 499 aa, Homo sapiens
MLLLLLLLPLLWGTKGMEGDRQYGDGYLLQVQELVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGAGATALAFLSFCIIFIIVRSCRKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 27 - protein, 463 aa, Homo sapiens
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQGPLTEPWAEDSPPDQPPPASARSSVGEGELQYASLSFQMVKPWDSRGQEATDTEYSEIKIHR

SEQ ID NO: 28 - protein, 639 aa, Homo sapiens
MLLPLLLSSLLGGSQAMDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSNGAFLGIGITALLFLCLALIIMKILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 29 - protein, 595 aa, Homo sapiens
MLLLLLLLPPLLCGRVGAKEQKDYLLTMQKSVTVQEGLCVSVLCSFSYPQNGWTASDPVHGYWFRAGDHVSRNIPVATNNPARAVQEETRDRFHLLGDPQNKDCTLSIRDTRESDAGTYVFCVERGNMKWNYKYDQLSVNVTASQDLLSRYRLEVPESVTVQEGLCVSVPCSVLYPHYNWTASSPVYGSWFKEGADIPWDIPVATNTPSGKVQEDTHGRFLLLGDPQTNNCSLSIRDARKGDSGKYYFQVERGSRKWNYIYDKLSVHVTALTHMPTFSIPGTLESGHPRNLTCSVPWACEQGTPPTITWMGASVSSLDPTITRSSMLSLIPQPQDHGTSLTCQVTLPGAGVTMTRAVRLNISYPPQNLTMTVFQGDGTASTTLRNGSALSVLEGQSLHLVCAVDSNPPARLSWTWGSLTLSPSQSSNLGVLELPRVHVKDEGEFTCRAQNPLGSQHISLSLSLQNEYTGKMRPISGVTLGAFGGAGATALVFLYFCIIFVVVRSCRKKSARPAVGVGDTGMEDANAVRGSASQGPLIESPADDSPPHHAPPALATPSPEEGEIQYASLSFHKARPQYPQEQEAIGYEYSEINIPK

SEQ ID NO: 30 - protein, 6 aa, Artificial Sequence
  Consensus sequence
XXYXXX

(19) United States
(12) Patent Application Publication
(10) Pub. No.: US 2003/0040604 A1
(43) Pub. Date: Feb. 27, 2003

(54) Siglec-12 polypeptides, polynucleotides, and methods of use thereof

(76) Inventors: Dirk M. Anderson, Seattle, WA (US); John S. Marken, Seattle, WA (US)

Correspondence Address: IMMUNEX CORPORATION, LAW DEPARTMENT, 51 UNIVERSITY STREET, SEATTLE, WA, 98101

(21) Appl. No.: 10/158,238

(22) Filed: May 29, 2002

Related U.S. Application Data
(60) Provisional application No. 60/294,199, filed on May 29, 2001.

(51) Int. Cl. C07K 14/705; C07H 21/04; C12P 21/02; C12N 5/06

(52) U.S. Cl. 530/350; 435/69.1; 435/320.1; 435/325; 536/23.2

(57) ABSTRACT

Provided herein are polypeptide and polynucleotide sequences for a molecule having homology to the siglec family of polypeptides. Also provided are methods of making and using a siglec-like polypeptide and polynucleotide.

### CROSS REFERENCE TO RELATED APPLICATIONS

This application claims the benefit of priority under 35 U.S.C. §119 to U.S. Provisional Application Serial No. 60/294,199, filed May 29, 2001, the disclosure of which is incorporated herein by references.

### FIELD OF THE INVENTION

The invention is directed to novel purified polypeptides of the siglec family and fragments thereof, polynucleotides encoding such polypeptides, processes for production of recombinant forms of such polypeptides, antibodies generated against these polypeptides or fragments, and assays and methods employing these polypeptides, antibodies, and polynucleotide.

### BACKGROUND

Sialic acid-binding immunoglobulin-like lectins, or “siglecs” are Type I membrane proteins that are classified by sequence homology as constituting a distinct group within the immunoglobulin (Ig) superfamily (for review, see Williams et al., Cold Spring Harbor Symposia on Quantitative Biology 54 (part 2):637-647, 1989). The siglecs also are referred to as the sialoadhesin family. Since the identification of a macrophage-specific sialoadhesin protein (Crocker et al., EMBO J. 13: 4490-503, 1994), many other proteins have been classified as members of the siglec protein family (see Crocker et al., Glycoconj. J. 14:601-609, 1997; Matthews et al., Leukemia 12 (suppl. 1):S33-S36, 1998).

Several siglec family members have been reported, including CD22 (Umansky et al., Immunology 87:303-309, 1996; and Umansky et al., J. Mol. Med. 74:353-363, 1996); CD33 (Freeman et al., Blood 85:2005-2012, 1995); the CD33-like proteins, CD33-L1 and CD33-L2 (Takei et al., Cytogenet Cell Genet 78:295-300, 1997); OBBP1 and OBBP2 (Patel et al., J Biol Chem 274:22729-38, 1999); SAF3 (EP 869 178); p75/AIRM1 (Falco et al., J Exp Med 190:793-801, 1999; Vitale et al., Proc Natl Acad Sci USA 96:15091-15096, 1999); SAF4 (WO 98/53840); the murine myelin-associated proteins, or “MAGs” (Fujita et al., Biochim. Biophys. Res. Com. 165:1162, 1989); avian Schwann cell myelin protein (SMP) (Dulac et al., Neuron 8:323, 1992); siglec-5 (Cornish et al., Blood 92:2123-32, 1998); siglec 7 (Nicoll et al., J Biol Chem 274:34089-34095, 1999); siglec 8 (Floyd et al., J. Biol. Chem. 275:861-866, 2000); and siglec 9 (Angata and Varki, J. Biol. Chem., 275(29):22127-22135, 2000). The CD33-L1 and CD33-L2 translation products appear to be transmembrane and secreted forms, respectively, of the same protein. SAF3, p75/AIRM1 and siglec 7 appear to encode essentially the same protein, and humacr70 (WO 9831799) appears to be an alternate form of this same protein. OBBP1 is essentially the same as CD33-L1, and OBBP2 appears to be the same as siglec 5, except for a few amino acid differences.

The elucidation of additional members of the siglec family can provide proteins useful for regulating the immune system and for controlling disorders associated with cells that express siglecs.

### SUMMARY OF THE INVENTION

The invention provides a substantially purified polypeptide comprising a Siglec-12 polypeptide, wherein the amino acid sequence of the Siglec-12 polypeptide is at least 80%, 90%, 95% or more identical to a sequence as set forth in SEQ ID NO:2, wherein the Siglec-12 polypeptide binds a sialic acid moiety. In one aspect the Siglec-12 polypeptide has a sequence from about amino acid 14 to 686 of SEQ ID NO:2. In another aspect the Siglec-12 polypeptide has a sequence from about amino acid 14 to 549 of SEQ ID NO:2.

The invention further provides a substantially purified polypeptide comprising a Siglec-12 extracellular domain, wherein the amino acid sequence of the Siglec-12 extracellular domain is at least 80% identical to a sequence as set forth from about amino acid 14 to 549 of SEQ ID NO:2, wherein the Siglec-12 extracellular domain binds a sialic acid moiety.

Also provided by the invention is a fusion polypeptide comprising a first polypeptide comprising an amino acid sequence as set forth from about amino acid 14 to 549 of SEQ ID NO:2 operably linked to a second polypeptide. In one aspect, the fusion polypeptide comprises an Fc polypeptide, a leucine zipper polypeptide, and/or a peptide linker.

The invention provides an isolated polynucleotide comprising a sequence selected from the group consisting of: (a) SEQ ID NO:1; (b) SEQ ID NO:1 from about nucleotide 40 to 2058; (c) SEQ ID NO:1 from about nucleotide 40 to 1647; (d) sequences complementary to SEQ ID NO:1; (e) sequences complementary to SEQ ID NO:1 from nucleotide 40 to 2058; (f) sequences complementary to SEQ ID NO:1 from nucleotide 40 to 1647; (g) any of a), b), c), d), e), or f) wherein T can also be U; and (h) fragments of (a)-(g) that are at least 50 bases in length and that will hybridize under moderate to highly stringent conditions to a nucleic acid which encodes a polypeptide consisting of a sequence as set forth in SEQ ID NO:2.

The invention includes a vector comprising a polynucleotide of the invention as well as host cells containing a vector of the invention.

The invention further provides a recombinant host cell comprising a polynucleotide of the invention under the control of a heterologous regulatory sequence. The host cell can be prokaryotic or eukaryotic.

The invention also provides a method of producing a polypeptide comprising culturing a host cell of claim of the invention under condition that promote expression of a Siglec-12 polypeptide.

Also provided by the invention are polypeptides produced by culturing a host cell of the invention under conditions that promote expression of a Siglec-12 polypeptide. The invention provides a substantially purified antibody that specifically binds to a polypeptide consisting of a sequence as set forth in SEQ ID NO:2. The antibody may be a monoclonal antibody, a polyclonal antibody, a human, or a humanized antibody.

Pharmaceutical compositions comprising an antibody and/or Siglec-12 polypeptide of the invention are also provided.

The invention also provides a method for identifying an agent which modulates expression of a polynucleotide comprising contacting a sample containing a polynucleotide comprising a sequence as set forth in SEQ ID NO:1 with a test agent and measuring the expression of the polynucleotide compared to a control, wherein a change in expression compared to the control is indicative of an agent that modulates expression of the polynucleotide.

The invention further provides a method for identifying an agent which modulates the activity of a polypeptide comprising contacting a sample containing a polypeptide comprising a sequence selected from the group consisting of (a) SEQ ID NO:2, (b) SEQ ID NO:2 from 14 to 686, and (c) SEQ ID NO:2 from 14 to 549, with a test agent and measuring the activity of the polypeptide compared to a control, wherein a change in activity compared to the control is indicative of an agent that modulates activity of the polypeptide.

The invention provides a method of treating a siglec-associated disorder or disease comprising contacting a subject with a Siglec-12 polypeptide or Siglec-12 polynucleotide in an amount effective to treat the siglec-associated disorder or disease.

The invention also provides a method of treating a subject having a tumor that expresses a Siglec-12 polypeptide, comprising administering to the subject an antibody that specifically binds a Siglec-12 polypeptide, wherein the antibody is conjugated to a radioisotope or toxin.

### BRIEF DESCRIPTION OF THE DRAWINGS

FIG. 1 shows an alignment of siglec 12 polypeptide (SEQ ID NO:2) with various members of the siglec family of polypeptides (Siglecs 3, 5, 6, 7, 8, 9, 10, and 11 (S2V; Zhenbao et al.) corresponding to SEQ ID Nos:22-29, respectively. Conserved cysteine residues are highlighted. The signal sequence and the transmembrane sequence are underlined. The putative ITIM and modified ITIM or SLAM sequences are highlighted. The first Ig domain is in bold, the second Ig domain is italicized, the third Ig domain is in reverse text, the fourth Ig domain is double underlined, and the fifth Ig domain is dotted underlined.

### DETAILED DESCRIPTION OF THE INVENTION

The invention provides polypeptides having homology to the siglec family of polypeptides. Also provided are polynucleotides encoding the novel siglec polypeptides as well as methods of use of the polynucleotides and polypeptides.

Siglecs have typically been characterized by sequence similarities and by their ability to bind to sialic acid moieties on glycoproteins and glycolipids. Generally, the extracellular portions of the siglecs are more similar, e.g., more highly conserved, than their cytoplasmic regions. The extracellular regions contain at least one V-set Ig-like domain located near the amino terminus followed by varying numbers of C2-set Ig-like domains. For example, CD33 has two Ig-like domains in its extracellular region, while sialoadhesin has 17. The siglecs—contain an unusual arrangement of conserved cysteine residues at their amino termini resulting in a predicted intra-β-sheet disulfide bridge in the first domain, and an interdomain disulfide bond between the first and eighth domains (see Crocker et al., 1997). Numerous siglec genes have been mapped to the same region of human chromosome 19.

The structural interactions between sialoadhesin and carbohydrates have been analyzed (for example, see Collins et al., J Biol Chem 272:16889-95, 1997; see also May et al., Mol. Cell 1:719-28, 1998). Siglecs exhibit functional protein-carbohydrate recognition through specific siaylated glycoconjugates on their cognate molecules, and some of them bind with glycans that terminate in α-2,3 linked sialic acids (Kelm et al., Curr. Biol. 4:965-72, 1994). The sialic acid-binding activity usually resides on the N-terminal V-set Ig-like domain, and may also involve the penultimate Ig-like domain. Some members of this group are reported to exhibit distinct specificities for both the type of sialic acid and its linkage to subterminal sugars.

Many proteins have been reported to contain a cytoplasmic inhibitory signaling motif that is associated with the transduction of inhibitory effector functions, e.g., the “immunoreceptor tyrosine-based inhibition motif,” or “ITIM” (Renard et al., Immun Rev 155:205-221, 1997). ITIMs have the consensus sequence I/VxYxxL/V (SEQ ID NO:30), and are found in the cytoplasmic portions of diverse signal transduction proteins of the immune system, many of which, like the siglecs, belong to the Ig superfamily or to the family of type II dimeric C-lectins (see Renard et al., 1997, supra). Proteins that contain ITIMs include the “killer cell Ig-like receptors,” or “KIRs,” and some members of the leukocyte Ig-like receptor or “LIR” family of proteins (Renard et al., 1997, supra; Cosman et al., Immunity 7:273-82, 1997; Borges et al., J Immunol 159:5192-96, 1997). The KIRs and LIRs, like the siglecs, are expressed on hematopoietic cells and map to chromosome 19. Signal transduction by an ITIM is believed to downregulate targeted cellular activities, such as expression of cell surface proteins. Renard et al. propose that the regulation of complex cellular functions is fine-tuned by the interplay of ITIM-mediated inhibitory signal transduction and activation of the same functions by a 16-18 amino acid activitory motif, or “ITAM” sequence that is present in other proteins.

Some of the siglecs have been reported to contain one or more ITIMs in their cytoplasmic regions. CD22 has more than one ITIM and has been characterized as a negative regulator of B cell activation. CD33 and siglec 8 also are reported to contain ITIM motifs in their cytoplasmic domains (Ulyanova et al., Eur J Immunol 29:3440-49, 1999; Floyd et al., 2000, supra). An ITIM is also present in the cytoplasmic tail of p75/AIRM1/siglec 7, a protein expressed at significant levels on a subset of CD8⁺ natural killer (NK) cells (Nicoll et al., 1999, supra). Falco et al. (1999, supra) have reported that downregulation of spontaneous NK-mediated cytotoxicity or NK-mediated cytotoxicity triggered via any of several activating receptors, could be brought about by cross-linking p75/AIRM1.

Siglec expression is restricted largely to myeloid cells of the immune system, and is believed to be involved in control of myeloid interactions, such as adhesions between antigen presenting cells (APCs), e.g., macrophages (including microglia) or dendritic cells, and other cells involved in cell-mediated immunity, such as T cells or natural killer cells. These polypeptides may function in antigen capture and uptake when expressed on APCs, and thus may provide targets for enhancing cell-based tumor vaccines. Many siglecs are observed to be expressed primarily on subsets of specific types of hematopoietic cells. CD33 expression is largely restricted to the myelomonocytic lineage, and is present on mature monocytes and tissue macrophages (Freeman et al., 1995, supra). CD22 is expressed primarily on B-cells, while siglec-8 is expressed specifically on eosinophilic granulocytes (Floyd et al., J. Biol. Chem. 275:861-866, 2000). Sialoadhesin is expressed at high levels on macrophages in chronic inflammatory conditions and in tumors, suggesting a role in host defense, and can mediate specific cell-substrate and cell-cell interactions in vitro (Crocker et al., 1994; Crocker et al., 1997, supra). Umansky et al. have reported that sialoadhesin-positive macrophages contribute to host resistance against metastasis of tumors, that these macrophages can function as antigen-presenting cells, and also that sialoadhesion expression is responsive to corticosteroids, lymphokines and cytokines (Umansky et al., 1996 and 1996). However, siglec expression is not entirely confined to hematopoietic cells. At least two siglecs are expressed in neuronal cells, including the avian SMP protein, which was first isolated from glial cells (Dulac et al.), and the MAGs that were isolated from a rat brain cDNA library (Fujita et al., 1989).

CD33 maps to a region of chromosome 19 that was associated with an interstitial deletion (del(9)(ql2-q22)) in several patients with acute myeloblastic leukemia (AML) or T-cell acute lymphoblastic leukemia (T-ALL) (Ferrara et al., Leukemia 10:1990-92, 1996). Two of these AML patients had exhibited a myelodysplastic syndrome prior to the onset of AML. Antibodies against CD33 are used in the diagnostic differentiation of myeloid leukemic cells from the more commonly occurring CD33-negative leukemias (e.g., see Freeman et al., 1995), and such antibodies also have been used with some success in the treatment of AML (Maloney et al., Curr. Opin. Hematol. 5: 237, 1998).

The invention provides a novel member of the siglec family of proteins, referred to herein as “Siglec-12”. A Siglec-12 polypeptide of the invention includes a polypeptide which contains or comprises an amino acid sequence as set forth in SEQ ID NO:2; polypeptides having substantial homology/identity to a sequences set forth in SEQ ID NO:2; fragments of the foregoing sequences (e.g., bioactive fragments); and conservative variants of the foregoing. The polypeptides of the invention have been shown to have homology to a number of siglec family polypeptides and thus have predicted function as siglec polypeptides.

As used herein, “polypeptide” means any chain of amino acids (including L- or D-amino acids), regardless of length or post-translational modification (e.g., glycosylation or phosphorylation), and include natural proteins, synthetic or recombinant polypeptides and fragments as well as a recombinant molecule consisting of a hybrid with a first portion, for example, having all or part of a Siglec-12 polypeptide amino acid sequence and a second portion comprising all or part of a polypeptide of interest. Typically, a Siglec-12 polypeptide is substantially pure of other components from which it is normally present in nature. The term “substantially pure” or “purified” when referring to a polypeptide, means a polypeptide that is at least 30% free from the proteins and naturally-occurring organic molecules with which it is naturally associated. Typically a substantially purified polypeptide of the invention is at least 35-50%, at least 60-70%, at least 75%, at least 90%, but will typically be at least 99% by weight purified from other naturally occurring organic molecules. A substantially purified polypeptide of the invention can be obtained, for example, by extraction from a natural source, by expression of a recombinant polynucleotide encoding the polypeptide, or by chemically synthesizing the polypeptide. Purity can be measured by any appropriate method, e.g., column chromatography, polyacrylamide gel electrophoresis, or HPLC analysis.

In general, a recombinant polypeptide or fragment can be purified from a host cell if not secreted, or from the medium or supernatant if soluble and secreted, followed by one or more rounds of concentration, salting-out, ion exchange, hydrophobic interaction, affinity purification or size exclusion chromatography. If desired, the culture medium first can be concentrated using a commercially available protein concentration filter, for example, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed, including various insoluble matrices comprising sulfopropyl or carboxymethyl groups. In addition, a chromatofocusing step or, alternatively, a hydrophobic interaction chromatography step can be employed. Suitable matrices can be phenyl or octyl moieties bound to resins. In addition, affinity chromatography with a matrix that selectively binds the recombinant protein can be employed. Examples of such resins employed are lectin columns, dye columns, and metal-chelating columns. Finally, one or more reversed-phase high performance liquid chromatography (RP-HPLC) steps employing hydrophobic RP-HPLC media, (e.g., silica gel or polymer resin having pendant methyl, octyl, octyldecyl or other aliphatic groups) can be employed to further purify the polypeptides. Some or all of the foregoing purification steps, in various combinations, are well known and can be employed to provide a substantially purified Siglec-12 polypeptide of the invention.

It is also possible to utilize an affinity column comprising a polypeptide-binding protein, such as a monoclonal antibody generated against a Siglec-12 polypeptide of the invention, to affinity-purify expressed Siglec-12 polypeptides. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the invention.

Accordingly, polypeptide-binding proteins, such as anti-polypeptide antibodies or other proteins that may interact with a polypeptide of the invention, can be bound to a solid phase support such as a column chromatography matrix or a similar substrate suitable for identifying, separating, or purifying cells that express polypeptides of the invention on their surface. Adherence of polypeptide-binding proteins of the invention to a solid phase contacting surface can be accomplished by any means, for example, magnetic microspheres can be coated with these polypeptide-binding proteins and held in the incubation vessel through a magnetic field. Suspensions of cell mixtures are contacted with the solid phase that has such polypeptide-binding proteins thereon. Cells having polypeptides of the invention on their surface bind to the fixed polypeptide-binding protein and unbound cells then are washed away. This affinity-binding method is useful for purifying, screening, or separating such polypeptide-expressing cells from solution. The cells can be released, for example, by using a preferably non-toxic enzyme that cleaves the cell-surface binding partner, or by effecting such release by modifying the composition of the buffer.

Alternatively, mixtures of cells suspected of containing Siglec-12 polypeptide-expressing cells of the invention can be incubated with a biotinylated polypeptide-binding protein, such as an anti-Siglec-12 polypeptide antibody. Sufficient binding usually occurs within about one hour, after which the mixture is then passed through a column packed with avidin-coated beads, to which the biotin moiety will bind with high affinity (see Berenson, et al J. Cell. Biochem., 10D:239, 1986). Unbound cells are washed free of the column, and bound cells are eluted according to conventional methods. This method can be used to isolate cells (e.g., macrophages and microglial cells) expressing membrane-bound Siglec-12 polypeptides.

When purifying polypeptides, the desired degree of purity will depend on the intended use of the polypeptide. A relatively high degree of purity is desired when the polypeptide is to be administered in vivo, for example. In such a case, the polypeptides typically are purified such that no bands corresponding to other proteins are detectable by SDS-polyacrylamide gel electrophoresis (SDS-PAGE). One skilled in the art will understand that multiple bands corresponding to the polypeptide may be visualized by SDS-PAGE, due to differential glycosylation, differential post-translational processing, and the like. Typically the polypeptide of the invention is purified to substantial homogeneity, as indicated by a single protein band upon analysis by SDS-PAGE. The band may be visualized by silver staining, Coomassie blue staining, or (if the protein is radiolabeled) by autoradiography.

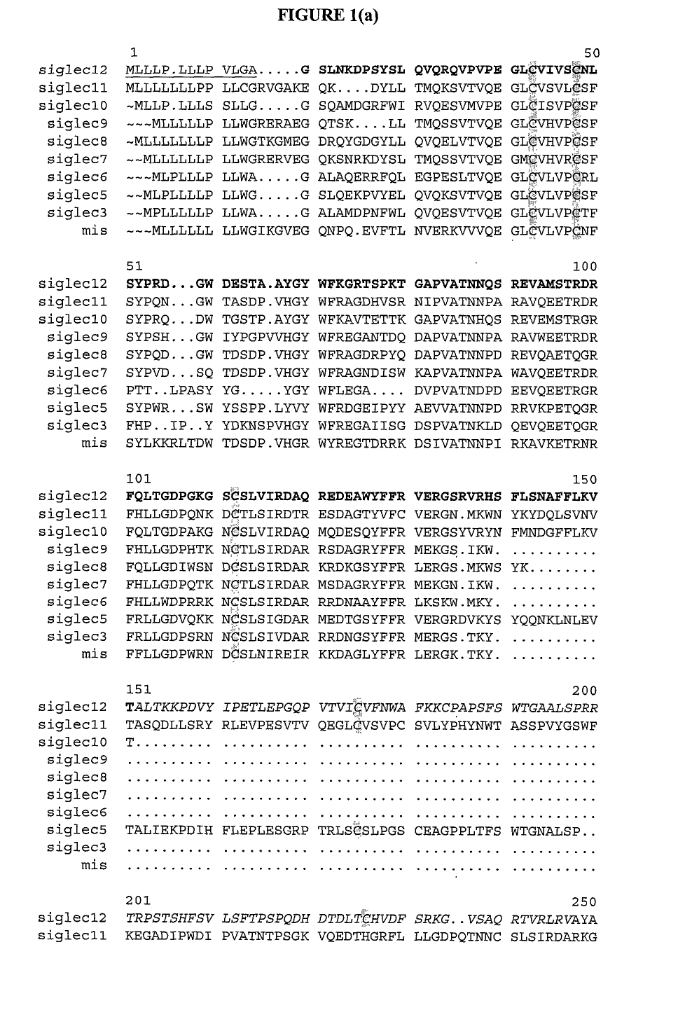

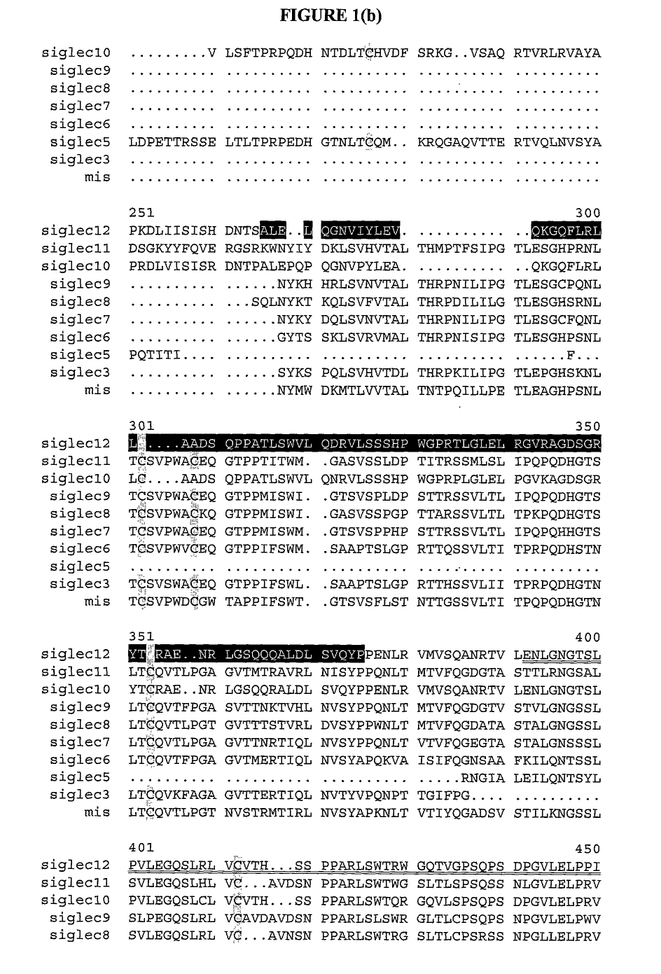

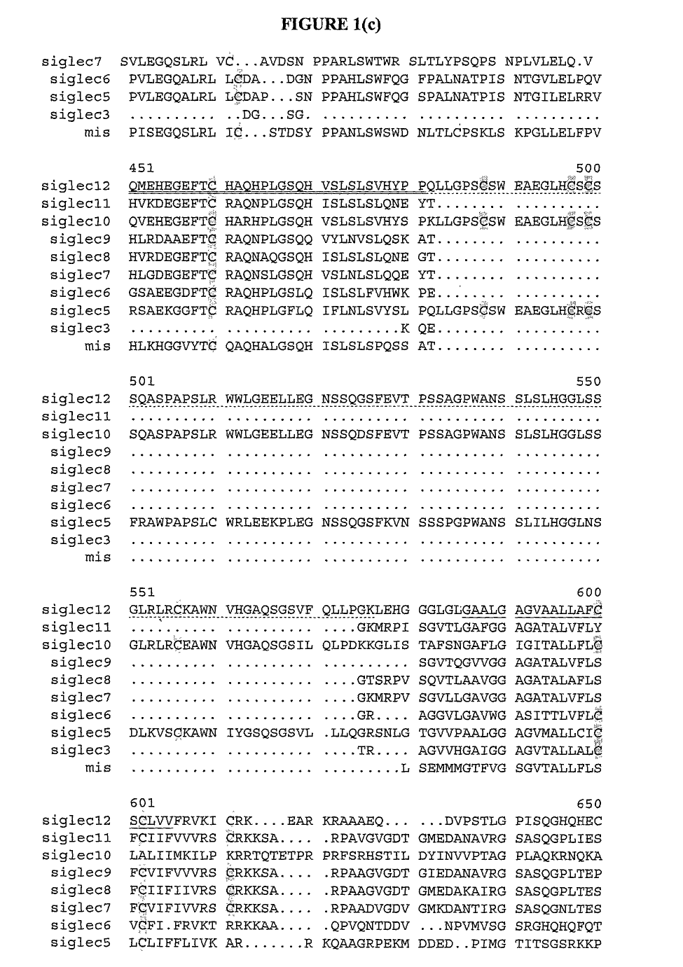

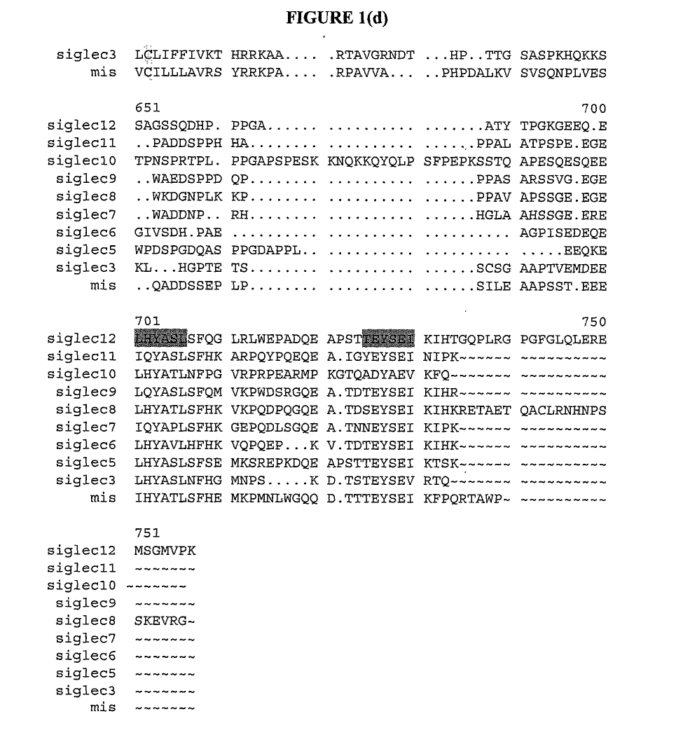

A Siglec-12 polypeptide of the invention comprises a number of distinct regions. A signal peptide, is present in Siglec-12. The signal peptide present in the full-length polypeptide of the invention is predicted to include amino acids 1-13 of SEQ ID NO:2. The signal peptide cleavage site for Siglec-12 polypeptide was predicted using a computer algorithm. However, one of skill in the art will recognize that the cleavage site of the signal peptide may vary depending upon a number of factors including the organism in which the polypeptide is expressed. Accordingly, the N-terminus of a mature form of a Siglec-12 polypeptide of the invention may vary by about 2 to 5 amino acids. Thus, a mature form of the Siglec-12 polypeptide of the invention may include at its N-terminus amino acids 9, 10, 11, 12, 13, 14, 15, 16, 17, 18, 19 or 20 of SEQ ID NO:2. Accordingly, a mature form of the Siglec-12 polypeptide includes amino acids 9, 10, 11, 12, 13, 14, 15, 16, 17, 18, 19 or 20 to about amino acid 686 (or, in the case of a soluble polypeptide, 549) of SEQ ID NO:2. An extracellular domain of a Siglec-12 polypeptide comprises from about amino acid 14 to 549 (including fragments thereof) of SEQ ID NO:2. The Ig-like domain assignments, as well as those for the transmembrane and cytoplasmic domains are based upon computer algorithms, on previous reports (Foussias et al., Genomics 67:171-178, 2000; Foussias et al., Biochem Biophys. Res. Comm. 278:775-781, 2000; Floyd et al., J. Biol. Chem. 275:861-866, 2000; and Munday et al., Biochem J. 355:489) and the one domain-one exon rule (Willams and Barclay, Annu. Rev. Immunol. 6:381405, 1988). The extracellular region of Siglec-12 polypeptide putatively contains five Ig-like domains located at about amino acids 14-141, 142-235, 253-340, 357-443, and 444-538 of SEQ ID NO:2. The transmembrane regions for these polypeptides are located at about amino acids 550 to 570 of SEQ ID NO:2. The intracellular regions are located at amino acids 571 to 686 of SEQ ID NO:2. The cytoplasmic portion of the Siglec-12 polypeptide contains a putative ITIM motif, as well as a second sequence that is a modified ITIM motif or a putative signaling lymphocyte activation molecule (SLAM) motif. The first of these has the sequence LHYASL (SEQ ID NO:3), and corresponds to amino acids 630 to 635 of SEQ ID NO:2. The second motif sequence is TEYSEI (SEQ ID NO:4), corresponding to amino acids 654 to 659 of SEQ ID NO:2. This second motif has homology to a sequence (TxYxx(IV)) recently found in the signaling lymphocyte activation molecule (SLAM) that is responsible for the binding of SLAM-associated protein (SAP) (Coffey et al., Nat. Genet. 20:129-135, 1998; Foussias et al., Genomics 67:171-178, 2000). Alternatively, the second motif may represent a functional variant of the ITIM motif. FIG. 1 shows the relative domains and conserved residues of Siglec-12 polypeptide indicative of a siglec polypeptide (see also, Angata et al., “Cloning and characterization of human Sigle-11. A recently evolved signaling molecule that can interact with SHP-1 and SHP-2 and is expressed by tissue macrophages, including brain microglia,” J. Biol. Chem., Papers in Press, e-published May 1, 2002 as Manuscript M202833200; which is incorporated herein in its entirety).

The invention provides both full-length and mature forms of Siglec-12 polypeptides. Full-length polypeptides are those having the complete primary amino acid sequence of the polypeptide as initially translated. The amino acid sequences of full-length polypeptides can be obtained, for example, by translation of the complete open reading frame (“ORF”) of a cDNA molecule. Several full-length polypeptides may be encoded by a single genetic locus if multiple mRNA forms are produced from that locus by alternative splicing or by the use of multiple translation initiation sites. An example of a full length Siglec-12 polypeptide of the invention comprises a sequence as set forth in SEQ ID NO:2 from amino acid 1 to amino acid 686. Such a full length polypeptide is contemplated to include, for example, the signal peptide comprising amino acids 1 to about amino acid 13 of SEQ ID NO:2.

A “mature form” of a polypeptide refers to a polypeptide that has undergone post-translational processing steps, if any, such as, for example, cleavage of the signal peptide or proteolytic cleavage to remove a prodomain. Multiple mature forms of a particular full-length polypeptide may be produced, for example, by imprecise cleavage of the signal sequence, or by differential regulation of proteases that cleave the polypeptide. The mature form(s) of such polypeptide may be obtained by expression, in a suitable mammalian cell or other host cell, of a polynucleotide that encodes the full-length polypeptide. The sequence of a mature form of the polypeptide may also be determinable from the amino acid sequence of the full-length form, through identification of signal peptides or protease cleavage sites (e.g., a protease cleavage site is predicted between the Ala-Gly residues at positions 13 and 14 of SEQ ID NO:2). An example of a mature form of a Siglec-12 polypeptide of the invention comprises a sequence as set forth in SEQ ID NO:2 from about amino acid 14 to about amino acid 686.

A Siglec-12 polypeptides of the invention also include polypeptides that result from post-transcriptional or post-translational processing events such as alternate mRNA processing which can yield a truncated but biologically active polypeptide, for example, a naturally occurring soluble form of the polypeptide. Also encompassed within the invention are variations attributable to proteolysis such as differences in the N- or C-termini upon expression in different types of host cells, due to proteolytic removal of one or more terminal amino acids from the polypeptide (generally from 1-5 terminal amino acids).

In another embodiment, the invention provides bioactive fragments of a Siglec-12 polypeptide. A bioactive fragment includes a fragment of SEQ ID NO:2 having a biological activity associated with a siglec polypeptide and/or a biological activity associated with a full-length or mature form of a Siglec-12 polypeptide of the invention. A biological activity associated with a bioactive fragment or a Siglec-12 polypeptide includes, for example, cell-cell interactions, cell adhesion, modulation of cytokine expression, modulation of calcium mobilization, or binding to sialic acid containing proteins (e.g., binding to α2-8-linked sialic acids). For example, ITIM domain containing proteins have been shown to inhibit calcium mobilization in cells (Fournier et al., J. Immunol. 165(3):1197-1209, 2000). Thus, for example, a bioactive fragment of the invention is a fragment that modulates calcium mobilization, modulates interactions with protein tyrosine phosphatases (e.g. SHP-1 and SHP-2), and/or modulate tyrosine phosphorylation. Examples of bioactive fragments of a Siglec-12 polypeptide molecules include those having a sequence as set forth in SEQ ID NO:2 from about amino acid 14 to 549 and fragments thereof (e.g., from about amino acid 14 to 141; from about amino acid 142 to 235; from about amino acid 253 to 340; from about amino acid 357 to 443; from about amino acid 444 to 538; from about amino acid 14 to 235; from about amino acid 14 to 340; from about amino acid 14 to 443; from about amino acid 14 to 538; from about amino acid 142 to 340; from about amino acid 142 to 443 of SEQ ID NO:2, and the like). Such bioactive fragments represent soluble molecules lacking the predicted transmembrane domain (e.g., the domain beginning at about amino acid 550 to amino acid 570 of SEQ ID NO:2). Bioactive fragments of Siglec-12 polypeptides are capable of interacting, for example, with a Siglec-12 polypeptide cognate, or with an antibody developed against a Siglec-12 polypeptide of SEQ ID NO:2, or inhibit the cross-linking of a native Siglec-12 with another native Siglec-12 thereby inhibiting dimerization. Methods of determining whether a Siglec-12 polypeptide or bioactive fragment of a Siglec-12 polypeptide of the invention has a desired activity can be accomplished by assaying the polypeptide by any of the methods described herein below as well as those disclosed in Angata et al., 2002, supra.

Accordingly, the polypeptides of the invention may be membrane-bound or they may be secreted and thus soluble. Soluble polypeptides are capable of being secreted from the cells in which they are expressed. In general, soluble polypeptides may be identified (and distinguished from non-soluble membrane-bound counterparts) by separating intact cells which express the desired polypeptide from the culture medium, e.g., by centrifugation, and assaying the medium (supernatant) for the presence of the desired polypeptide. The presence of polypeptide in the medium indicates that the polypeptide was secreted from the cells and thus is a soluble form of the polypeptide.

In one embodiment, the soluble polypeptides (e.g., a bioactive fragment of a Siglec-12 polypeptide) comprise all or part of the extracellular domain, but lack the transmembrane region that would cause retention of the polypeptide in a cell membrane. A soluble polypeptide according to the invention may include the cytoplasmic domain, or a portion thereof, so long as the polypeptide is secreted from the cell in which it is produced.

In general, the use of soluble forms is advantageous for certain applications. Purification of the polypeptides from recombinant host cells is facilitated, since the soluble polypeptides are secreted from the cells. Further, soluble polypeptides are generally more suitable for intravenous administration. A soluble form of a Siglec-12 polypeptide of the invention comprising, for example, the extracellular domain of a Siglec-12 polypeptide find uses in binding to a native Siglec-12 polypeptide thereby inhibiting dimerization with another native Sigle-12, and/or binding to a Siglec-12 binding partner thereby inhibiting binding of the native molecule to the binding partner. In addition, a soluble form of a Siglec-12 polypeptide may bind to an activate a Siglec-12 polypeptide by forming a dimer with a native Siglec-12 thereby inducing a biological activity related indicative of dimerization between two native Siglec-12 polypeptides, and/or may bind to a Siglec-12 binding partner, thus inducing a biological activity related to binding of a native Siglec-12 polypeptide to its binding partner.

The invention also provides polypeptides and fragments of the extracellular domain that retain the capacity to bind a sialic acid containing moiety (e.g., a α2-8 sialic acid moiety), modulate calcium mobilization, or bind to a Siglec-12 polypeptide cognate and thereby inhibit binding by the native Siglec-12 polypeptide to its cognate. Such a fragment may be a soluble polypeptide, as described above.

Also provided herein are polypeptide fragments comprising at least 50, or at least 60, contiguous amino acids of the sequence of SEQ ID NO:2. Fragments derived from the cytoplasmic domain find use in studies of signal transduction, and in regulating cellular processes associated with transduction of biological signals, such as inhibitory signals, and in identifying small molecule mimics or inhibitors of receptor interaction with signaling molecules. For example, a Siglec-12 polypeptide cytoplasmic domain (e.g., from about amino acid 571 to 686 of SEQ ID NO:2) can be used to modulate intracellular phosphorylation and/or SHP-1 and SHP-2 activity. In one embodiment, a polynucleotide encoding a polypeptide comprising the cytoplasmic domain of Siglec-12 is expressed in a cell.

In another embodiment, fragments of a Siglec-12 polypeptide comprising at least 8-11, or more preferably 10-30, contiguous amino acids of SEQ ID NO:2 specific to Siglec-12 may be employed as immunogens for generating antibodies.

Naturally occurring variants as well as derived variants of the disclosed polypeptides and fragments are provided herein. Variants may exhibit amino acid sequences that are at least 80% identical to the disclosed polypeptides and fragments. Also provided are polypeptides or fragments comprising an amino acid sequence that is at least 85% identical, at least 90% identical, at least 95% identical, at least 98% identical, at least 99% identical, or at least 99.9% identical to the amino acid sequences disclosed herein. In another aspect, the invention provides a Siglec-12 polypeptide variant having an amino acid sequence that varies by 1-10 conservative amino acid substitutions, 1-10 amino acid deletions, and/or 1-10 amino acid insertions compared with a Siglec-12 polypeptide having a sequence as set forth in SEQ ID NO:2.

Percent homology/identity may be determined by visual inspection and mathematical calculation. Alternatively, the percent homology/identity of two protein sequences can be determined by comparing sequence information using the a computer program, such as the GAP program, based on the algorithm of Needleman and Wunsch (J. Mol. Bio. 48:443, 1970) and available from the University of Wisconsin Genetics Computer Group (UWGCG). The preferred default parameters for the GAP program include: (1) a scoring matrix, blosum62, as described by Henikoff and Henikoff (Proc. Natl. Acad. Sci. USA 89:10915, 1992); (2) a gap weight of 12; (3) a gap length weight of 4; and (4) no penalty for end gaps. Similar comparison parameters can be implemented using other computer programs such as, for example, BESTFIT, FASTA, TFASTA (see, e.g., Wisconsin Genetics Software Package, Genetics Computer Group, 575 Science Dr., Madison, Wis.), or PILEUP (a simplification of the progressive alignment method of Feng & Doolittle, J. Mol. Evol. 35:351-360 (1987)).

The variants of the invention include, for example, those that result from alternate mRNA splicing events or from proteolytic cleavage. Alternate splicing of mRNA may, for example, yield a truncated but biologically active protein, such as a naturally occurring soluble form of the protein. Variations attributable to proteolysis include, for example, differences in the N- or C-termini upon expression in different types of host cells, due to proteolytic removal of one or more terminal amino acids from the protein (generally from 1-5 terminal amino acids). Proteins in which differences in amino acid sequence are attributable to genetic polymorphism (allelic variation among individuals producing the protein) are also contemplated herein.

Additional variants within the scope of the invention include polypeptides that may be modified to create derivatives thereof by forming covalent or aggregative conjugates with other chemical moieties, such as glycosyl groups, lipids, phosphate, acetyl groups and the like. Covalent derivatives may be prepared by linking the chemical moieties to functional groups on amino acid side chains or at the N-terminus or C-terminus of a polypeptide. Conjugates comprising diagnostic (detectable) or therapeutic agents attached thereto are contemplated herein, as discussed in more detail below.

Other derivatives include covalent or aggregative conjugates of the polypeptides with other proteins or polypeptides, such as by synthesis in recombinant culture as N-terminal or C-terminal fusions. Examples of fusion polypeptides are discussed below in connection with oligomers. Further, fusion polypeptides can comprise peptides added to facilitate purification and identification. Such peptides include, for example, poly-His or the antigenic identification peptides described in U.S. Pat. No. 5,011,912 and in Hopp et al., Bio/Technology 6:1204, 1988. One such peptide is the FLAG® peptide, Asp-Tyr-Lys-Asp-Asp-Asp-Asp-Lys (SEQ ID NO:5), which is highly antigenic and provides an epitope reversibly bound by a specific monoclonal antibody, enabling rapid assay and facile purification of expressed recombinant protein. A murine hybridoma designated 4E11 produces a monoclonal antibody that binds the FLAG® peptide in the presence of certain divalent metal cations, as described in U.S. Pat. No. 5,011,912, hereby incorporated by reference. The 4E11 hybridoma cell line has been deposited with the American Type Culture Collection under accession no. HB 9259. Monoclonal antibodies that bind the FLAG® peptide are available from Eastman Kodak Co., Scientific Imaging Systems Division, New Haven, Conn.

Among the variant polypeptides provided herein are variants of native Siglec-12 polypeptides that retain the native binding properties of a mature Siglec-12 polypeptide of SEQ ID NO:2 or the substantial equivalent thereof. One example is a variant that binds its binding partner with essentially the same binding affinity, as does the native form. Binding affinity can be measured by conventional procedures, e.g., as described in U.S. Pat. No. 5,512,457 and as set forth below.

Variants include polypeptides that are substantially homologous to the native form, but which have an amino acid sequence different from that of the native form because of one or more deletions, insertions or substitutions. Particular embodiments include, but are not limited to, polypeptides that comprise from one to ten deletions, insertions, or substitutions of amino acid residues, when compared to a native sequence.

A given amino acid may be replaced, for example, by a residue having similar physiochemical characteristics. Examples of such conservative substitutions include substitution of one aliphatic residue for another, such as Ile, Val, Leu, or Ala for one another; substitutions of one polar residue for another, such as between Lys and Arg, Glu and Asp, or Gln and Asn; or substitutions of one aromatic residue for another, such as Phe, Trp, or Tyr for one another. Other conservative substitutions, e.g., involving substitutions of entire regions having similar hydrophobicity characteristics, are well known.

Similarly, the polynucleotides of the invention include variants that differ from a native Siglec-12 polynucleotide because of one or more deletions, insertions or substitutions, but that encode a biologically active polypeptide, e.g., variants that exhibit inhibitory activity, interact with proteins having α2-8 sialic acid moieties, and the like.

Sialoadhesins contain a number of potential glycosylation sites. The invention further includes polypeptides of the invention with or without associated native-pattern glycosylation. Polypeptides expressed in yeast or mammalian expression systems (e.g., COS-1 or COS-7 cells) can be similar to or significantly different from a native polypeptide in molecular weight and glycosylation pattern, depending upon the choice of expression system. Expression of any of the polypeptides of the invention in bacterial expression systems, such as E. coli, provides non-glycosylated forms of the polypeptides. Further, a given preparation may include multiple differentially glycosylated species of the protein. Glycosyl groups can be removed through conventional methods, in particular those utilizing glycopeptidase. In general, glycosylated polypeptides of the invention can have their carbohydrate moieties removed by being incubated with a molar excess of glycopeptidase (Boehringer Mannheim).

N-glycosylation sites in eukaryotic polypeptides are characterized by an amino acid triplet Asn-X-Y, wherein X is any amino acid except Pro and Y is Ser or Thr. The Siglec-12 polypeptides of the invention have a number of putative glycosylations sites. For example, the Asn residue at one or more of the following positions is a potential glycosylation site: 43N, 78N, 250N, 354N, 363N, 485N, and 503N of SEQ ID NO:2. N-glycosylation sites in the polypeptide extracellular domain can be modified to preclude glycosylation, allowing expression of a reduced carbohydrate analog in mammalian and yeast expression systems. Accordingly, modifications (e.g., treatment with a glycopeptidase) or substitutions or deletions of these residues can modulate the activity of a mature Siglec-12 polypeptide of the invention.

Correspondingly, similar polynucleotide constructs that encode various additions or substitutions of amino acid residues or sequences, or deletions of terminal or internal residues or sequences are encompassed by the invention. Appropriate substitutions, additions, or deletions to the nucleotide sequence encoding these triplets (e.g., Asn-X-Y) will result in prevention of attachment of carbohydrate residues at the Asn side chain. Alteration of a single nucleotide, chosen so that Asn is replaced by a different amino acid, for example, is sufficient to inactivate an N-glycosylation site. Alternatively, a Ser or Thr in the triplet can by replaced with another amino acid, such as Ala. Known procedures for inactivating N-glycosylation sites in proteins include those described in U.S. Pat. No. 5,071,972 and EP 276,846. One of skill in the art can identify the corresponding nucleotide sequence corresponding to the putative glycosylation sites based upon the identified Asn residues identified above (e.g., 43N) with reference, for example, to the coding sequence provided in SEQ ID NO:1.

In another example of variants, sequences encoding Cys residues that are not essential for biological activity can be altered to cause the Cys residues to be deleted or replaced with other amino acids, preventing formation of incorrect intramolecular disulfide bridges upon folding or renaturation. A number of putative conserved Cys residues of the Siglec-12 polypeptides of the invention are identified in the alignment provided in FIG. 1.

Other variants are prepared by modification of adjacent dibasic amino acid residues, to enhance expression in yeast systems in which KEX2 protease activity is present. EP 212,914 discloses the use of site-specific mutagenesis to inactivate KEX2 protease processing sites in a protein. KEX2 protease processing sites are inactivated by deleting, adding or substituting residues to alter Arg-Arg, Arg-Lys, and Lys-Arg pairs to eliminate the occurrence of these adjacent basic residues. Lys-Lys pairings are considerably less susceptible to KEX2 cleavage, and conversion of Arg-Lys or Lys-Arg to Lys-Lys represents a conservative and preferred approach to inactivating KEX2 sites.

Oligomers

Encompassed by the invention are oligomers and fusion polypeptides, that comprise a Siglec-12 polypeptide or a bioactive fragment thereof. In one embodiment, the fusion partner is linked to the C-terminus of the Siglec-12 polypeptide or a bioactive fragment thereof. Such oligomers may be in the form of covalently-linked or non-covalently-linked multimers, including dimers, trimers, or higher oligomers. As noted above, soluble Siglec-12 polypeptides are provided and thus oligomers may comprise soluble Siglec-12 polypeptides. In one aspect of the invention, the oligomers maintain the binding ability of the polypeptide components and provide therefor, bivalent, trivalent, and the like, binding sites.

One embodiment of the invention is directed to oligomers comprising multiple polypeptides joined via covalent or non-covalent interactions between peptide moieties fused to the polypeptides. Such peptide moieties may be peptide linkers (spacers), or peptides that have the property of promoting oligomerization. Examples of peptide linkers include -Gly-Gly-, GGGGS (SEQ ID NO:6) (GGGGS)ₙ (SEQ ID NO:7), GKSSGSGSESKS (SEQ ID NO:8), GSTSGSGKSSEGKG (SEQ ID NO:9), GSTSGSGKSSEGSGSTKG (SEQ ID NO:10), GSTSGSGKPGSGEGSTKG (SEQ ID NO:11), or EGKSSGSGSESKEF (SEQ ID NO:12). Linking moieties are described, for example, in Huston, J. S., et al., PNAS 85:5879-5883 (1988), Whitlow, M., et al., Protein Engineering 6:989-995 (1993), and Newton, D. L., et al., Biochemistry 35:545-553 (1996). Other suitable peptide linkers are those described in U.S. Pat. Nos. 4,751,180 and 4,935,233, that are hereby incorporated by reference. A polynucleotide encoding a desired peptide linker can be inserted between, and in the same reading frame as, a polynucleotide encoding a Siglec-12 polypeptide of bioactive fragment of the invention, using any suitable conventional technique. In particular embodiments, a fusion polypeptide comprises from two to four bioactive fragments of a Siglec-12 polypeptide (e.g., a soluble fragment), separated by peptide linkers. In another embodiment, the invention provides a fusion polypeptide having an Fc polypeptide domain and a bioactive fragment as set forth in SEQ ID NO:2 from about amino acid 15 to 481, or fragment thereof. In one embodiment, the Fc fusion construct comprises amino acids 15 to 475 of SEQ ID NO:2 (encoded by nucleotides 40 to 1465). The Fc domains lead to the formation of oligomers comprising two or more Siglec-12 polypeptide domains. Leucine zippers and certain polypeptides derived from antibodies are among the peptides that can promote oligomerization of the polypeptides attached thereto, as described in more detail below.

As one alternative, an oligomer/fusion polypeptide is prepared using polypeptides derived from immunoglobulins. Preparation of fusion polypeptides comprising certain heterologous polypeptides fused to various portions of antibody-derived polypeptides (including the Fc domain) has been described, e.g., by Ashkenazi et al. (PNAS USA 88:10535, 1991); Byrn et al. (Nature 344:677, 1990); and Hollenbaugh and Aruffo (“Construction of Immunoglobulin Fusion Proteins”, in Current Protocols in Immunology, Suppl. 4, pages 10.19.1-10.19.11, 1992).

One embodiment of the invention is directed to a dimer comprising two fusion proteins created by fusing a Siglec-12 polypeptide or bioactive fragment of the invention to an Fc polypeptide derived from an antibody. A gene fusion encoding the Siglec-12-polypeptide/Fc fusion protein is inserted into an appropriate expression vector. The Siglec-12-polypeptide/Fc fusion proteins are expressed in host cells transformed with the recombinant expression vector, and allowed to assemble much like antibody molecules, whereupon interchain disulfide bonds form between the Fc moieties to yield divalent molecules.

An Fc polypeptide includes native and mutein forms of polypeptides made up of the Fc region of an antibody comprising any or all of the CH domains of the Fc region. Truncated forms of such polypeptides containing the hinge region that promotes dimerization are also included. In one aspect polypeptides comprise an Fc polypeptide derived from a human IgG1 antibody. The Fc polypeptides are linked to the COOH-terminus of a Siglec-12 polypeptide or bioactive fragment of the invention.

One suitable Fc polypeptide, described in PCT application WO 93/10151 (hereby incorporated by reference), is a single chain polypeptide extending from the N-terminal hinge region to the native C-terminus of the Fc region of a human IgGI antibody. Another useful Fc polypeptide is the Fc mutein described in U.S. Pat. No. 5,457,035 and in Baum et al., (EMBO J. 13:3992-4001, 1994) incorporated herein by reference. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

The above-described fusion proteins comprising Fc moieties (and oligomers formed therefrom) offer the advantage of facile purification by affinity chromatography over Protein A or Protein G columns.

In other embodiments, the polypeptides of the invention may be substituted for the variable portion of an antibody heavy or light chain. If fusion proteins are made with both heavy and light chains of an antibody, it is possible to form an oligomer with as many as four Siglec-12 polypeptide extracellular regions.

Another method for preparing the oligomers of the invention involves use of a leucine zipper. Leucine zipper domains are peptides that promote oligomerization of the proteins in which they are found. Leucine zippers were originally identified in several DNA-binding proteins (Landschulz et al., Science 240:1759, 1988), and have since been found in a variety of different proteins. Among the known leucine zippers are naturally occurring peptides and derivatives thereof that dimerize or trimerize.

The zipper domain (also referred to herein as an oligomerizing, or oligomer-forming, domain) comprises a repetitive heptad repeat, often with four or five leucine residues interspersed with other amino acids. Examples of zipper domains are those found in the yeast transcription factor GCN4 and a heat-stable DNA-binding protein found in rat liver (C/EBP; Landschulz et al., Science 243:1681, 1989). Two nuclear transforming proteins, fos and jun, also exhibit zipper domains, as does the gene product of the murine proto-oncogene, c-myc (Landschulz et al., Science 240:1759, 1988). The products of the nuclear oncogenes fos and jun comprise zipper domains that form heterodimer (O'Shea et al., Science 245:646, 1989, Turner and Tjian, Science 243:1689, 1989).

The fusogenic proteins of several different viruses, including paramyxovirus, coronavirus, measles virus and many retroviruses, also possess zipper domains (Buckland and Wild, Nature 338:547,1989; Britton, Nature 353:394, 1991; Delwart and Mosialos, AIDS Research and Human Retroviruses 6:703, 1990). The zipper domains in these fusogenic viral proteins are near the transmembrane region of the proteins; it has been suggested that the zipper domains could contribute to the oligomeric structure of the fusogenic proteins. Oligomerization of fusogenic viral proteins is involved in fusion pore formation (Spruce et al., Proc. Natl. Acad. Sci. U.S.A. 88:3523, 1991). Zipper domains have also been reported to play a role in oligomerization of heat-shock transcription factors (Rabindran et al., Science 259:230, 1993).

Zipper domains fold as short, parallel coiled coils (O'Shea et al., Science 254:539, 1991). The general architecture of the parallel coiled coil has been well characterized, with a “knobs-into-holes” packing as proposed by Crick in 1953 (Acta Crystallogr. 6:689). The dimer formed by a zipper domain is stabilized by the heptad repeat, designated (abcdefg)ₙ according to the notation of McLachlan and Stewart (J. Mol. Biol. 98:293; 1975), in which residues a and d are generally hydrophobic residues, with d being a leucine, which line up on the same face of a helix. Oppositely-charged residues commonly occur at positions g and e. Thus, in a parallel coiled coil formed from two helical zipper domains, the “knobs” formed by the hydrophobic side chains of the first helix are packed into the “holes” formed between the side chains of the second helix.

The residues at position d (often leucine) contribute large hydrophobic stabilization energies, and are important for oligomer formation (Krystek: et al., Int. J Peptide Res. 38:229, 1991). Lovejoy et al. (Science 259:1288, 1993) reported the synthesis of a triple-stranded α-helical bundle in which the helices run up-up-down. Their studies confirmed that hydrophobic stabilization energy provides the main driving force for the formation of coiled coils from helical monomers. These studies also indicate that electrostatic interactions contribute to the stoichiometry and geometry of coiled coils. Further discussion of the structure of leucine zippers is found in Harbury et al. (Science 262:1401, 26 November 1993).

Examples of leucine zipper domains suitable for producing soluble oligomeric proteins are described in PCT application WO 94/10308, as well as the leucine zipper derived from lung surfactant protein D (SPD) described in Hoppe et al. (FEBS Letters 344:191, 1994), hereby incorporated by reference. The use of a modified leucine zipper that allows for stable trimerization of a heterologous protein fused thereto is described in Fanslow et al. (Semin. Immunol. 6:267-278, 1994). Recombinant fusion proteins comprising a bioactive fragment of the invention (e.g., a soluble fragment) fused to a leucine zipper peptide are expressed in suitable host cells, and the soluble oligomer that forms is recovered from the culture supernatant.

Certain leucine zipper moieties form trimers. One example is a leucine zipper derived from lung surfactant protein D (SPD) noted above, as described in Hoppe et al. and in U.S. Pat. No. 5,716,805, hereby incorporated by reference in their entirety. This lung SPD-derived leucine zipper peptide comprises the amino acid sequence Pro-Asp-Val-Ala-Ser-Leu-Arg-Gln-Gln-Val-Glu-Ala-Leu-Gln-Gly-Gln-Val-Gln-His-Leu-Gln-Ala-Ala-Phe-Ser-Gln-Tyr (SEQ ID NO:13).

Another example of a leucine zipper that promotes trimerization is a peptide comprising the amino acid sequence Arg-Met-Lys-Gln-Ile-Glu-Asp-Lys-Ile-Glu-Glu-Ile-Leu-Ser-Lys-Ile-Tyr-His-Ile-Glu-Asn-Glu-Ile-Ala-Arg-Ile-Lys-Lys-Leu-Ile-Gly-Glu-Arg (SEQ ID NO:14), as described in U.S. Pat. No. 5,716,805. In one alternative embodiment, an N-terminal Asp residue is added; in another, the peptide lacks the N-terminal Arg residue.

Fragments of the foregoing zipper peptides that retain the property of promoting oligomerization may be employed as well. Examples of such fragments include, but are not limited to, peptides lacking one or two of the N-terminal or C-terminal residues presented in the foregoing amino acid sequences. Leucine zippers may be derived from naturally occurring leucine zipper peptides, e.g., via conservative substitution(s) in the native amino acid sequence, wherein the peptide's ability to promote oligomerization is retained. In particular embodiments, leucine residues in a leucine zipper moiety are replaced by isoleucine residues. Such peptides comprising isoleucine may be referred to as isoleucine zippers, but are encompassed by the term “leucine zippers” as employed herein.

Antibodies

The polypeptides, fragments (e.g., soluble or bioactive fragments), variants, fusion proteins, and the like, as set forth above may be employed as “immunogens” in producing antibodies immunoreactive therewith. More specifically, the polypeptides, fragment, variants, fusion proteins, and the like, contain antigenic determinants or epitopes that elicit the formation of antibodies. Suitable antigenic determinants or epitopes may be either linear or conformational (discontinuous). Linear epitopes are composed of a linear series of amino acids linked to one another by covalent bonds, while conformational or discontinuous epitopes are composed of amino acids sections from different regions of the polypeptide chain that are brought into close proximity upon protein folding (Janeway and Travers, Immuno Biology 3:9 (Garland Publishing Inc., 2nd ed. 1996)). Because folded proteins have complex surfaces, the number of epitopes available is quite numerous; however, due to the conformation of the protein and steric hindrances, the number of antibodies that actually bind to the epitopes is less than the number of available epitopes (Janeway and Travers, Immuno Biology 2:14 (Garland Publishing Inc., 2nd ed. 1996)). Epitopes may be identified by methods known in the art.

The epitopes derived from the disclosed polypeptides are useful for raising antibodies, including monoclonal antibodies, and can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

The polyclonal and monoclonal antibodies elicited by the disclosed polypeptides, whether the epitopes have been isolated or remain part of the polypeptides, may be prepared by conventional techniques. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988).

Hybridoma cell lines that produce monoclonal antibodies specific for the polypeptides of the invention are also contemplated herein, and may be produced and identified by conventional techniques. One method for producing such a hybridoma cell line comprises immunizing an animal with a polypeptide; harvesting spleen cells from the immunized animal; fusing said spleen cells to a myeloma cell line, thereby generating hybridoma cells; and identifying a hybridoma cell line that produces a monoclonal antibody that binds the polypeptide. The monoclonal antibodies may be recovered by conventional techniques.

The monoclonal antibodies of the invention include chimeric antibodies, e.g., humanized versions of murine monoclonal antibodies. Such humanized antibodies may be prepared by known techniques and offer the advantage of reduced immunogenicity when the antibodies are administered to humans, such as for therapeutic purposes. In one embodiment, a humanized monoclonal antibody comprises the variable region of a murine antibody (or just the antigen-binding site thereof) and a constant region derived from a human antibody. Alternatively, a humanized antibody fragment may comprise the antigen-binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, May, 1993).

A method for producing an antibody comprises immunizing a non-human animal, such as a transgenic mouse, with a Siglec-12 polypeptide or fragment thereof, whereby antibodies directed against the polypeptide or fragment are generated in the animal. Procedures have been developed for generating human antibodies in non-human animals. The antibodies may be partially human, or preferably completely human. For example, transgenic mice into which genetic material encoding one or more human immunoglobulin chains has been introduced may be employed. Such mice may be genetically altered in a variety of ways. The genetic manipulation may result in human immunoglobulin polypeptide chains replacing endogenous immunoglobulin chains in at least some (preferably virtually all) antibodies produced by the animal upon immunization. Procedures to generate antibodies transgenically can be found in GB 2,272,440, U.S. Pat. Nos. 5,814,318, 5,569,825 and 5,545,806 and related patents claiming priority therefrom, all of which are incorporated by reference herein. Typically, for use in humans, the antibodies are human; techniques for creating such human antibodies are also known and transgenic mice useful for making human antibodies are commercially available from, for example, Medarex Inc. (Princeton, N.J.) and Abgenix Inc. (Fremont, Calif.).

Expression of a humanized immunoglobulin sequences in bacterial hosts may be used to select higher affinity humanized immunoglobulin sequences by mutagenizing the CDR regions and producing bacteriophage display libraries which may be screened for humanized immunoglobulin CDR variants which possess high affinity and/or high specificity binding to a Siglec-12 polypeptide or fragment thereof. One potential advantage of such affinity sharpening is the generation of humanized immunoglobulin CDR variants that have improved binding affinity and/or reduced cross-reactivity with molecules other than a Siglec-12 polypeptide or fragment thereof. Methods for producing phage display libraries having immunoglobulin variable region sequences are provided in the art, for example, see Cesareni, FEBS Lett 307:66-70 (1992); Swimmer et al., Proc. Natl. Acad. Sci. USA 89:3756-60 (1992); Gram et al., Proc. Natl. Acad. Sci. USA 89:3576-80 (1992); Clackson et al., Nature 352:624-8 (1991); Scott & Smith, Science 249:386-90 (1990); Garrard et al., Bio/Techniques 9:1373-1377 (1991), which are incorporated herein by reference in their entirety for all purposes. The resultant affinity sharpened CDR variant humanized immunoglobulin sequences are subsequently expressed in a suitable host.

A further approach for obtaining human anti-Siglec-12 polypeptide antibodies is to screen a DNA library from human B cells according to the general protocol outlined by Huse et al., Science 246:1275-1281 (1989). Antibodies binding to a Siglec-12 polypeptide or fragment thereof are selected. Sequences encoding such antibodies (or binding fragments) are then cloned and amplified. The protocol described by Huse is rendered more efficient in combination with phage-display technology. See, e.g., Dower et al., WO 91/17271 and McCafferty et al., WO 92/01047 (each of which is incorporated by reference in its entirety for all purposes). In these methods, libraries of phage are produced in which members display different antibodies on their outer surfaces. Antibodies are usually displayed as Fv or Fab fragments. Phage displaying antibodies with a desired specificity are selected by affinity enrichment to a Siglec-12 polypeptide or fragment thereof.

Antigen-binding fragments of the antibodies, which may be produced by conventional techniques, are also encompassed by the invention. Examples of such fragments include, but are not limited to, scFv, Fab and F(ab′)₂ fragments. Antibody fragments and derivatives produced by genetic engineering techniques are also provided.

The antibodies of the invention can be used in assays to detect the presence of the polypeptides or fragments of the invention, either in vitro or in vivo. The antibodies also may be employed in purifying polypeptides or fragments of the invention by immunoaffinity chromatography.

Those antibodies that block binding of the polypeptides of the invention to their binding partners may be used to inhibit a biological activity that results from such binding. Such blocking antibodies may be identified using any suitable assay procedure, such as by testing antibodies for the ability to inhibit binding of a Siglec-12 polypeptide or bioactive fragment thereof to certain cells expressing the binding partners or cognates of such polypeptide or fragment. Alternatively, blocking antibodies may be identified in assays for the ability to inhibit a biological effect that results from binding of the polypeptides of the invention to target cells. Antibodies may be assayed for the ability to inhibit Siglec-12 polypeptide-mediated cellular activities, for example.

Such antibodies may be employed in in vitro procedures, or administered in vivo to inhibit a biological activity mediated by the polypeptide to which the antibody binds. Disorders caused or exacerbated (directly or indirectly) by the interaction of the polypeptides of the invention with cell surface (binding partner) receptor thus may be treated. A therapeutic method involves in vivo administration of a blocking antibody to a mammal in an amount effective in inhibiting a Siglec-12 polypeptide-mediated biological activity. Monoclonal antibodies are generally preferred for use in such therapeutic methods. In one embodiment, an antigen-binding antibody fragment is employed.

Antibodies may be screened for agonistic (e.g., Siglec-12 polypeptide-mimicking) properties or antagonistic properties. Such antibodies upon binding to a Siglec-12 polypeptide can induce the biological activity of Siglec-12 polypeptide including, for example, causing inhibition of cell activation or calcium mobilization. For example, the antibody can induce biological effects (e.g., transduction of biological signals) similar to the biological effects induced when a Siglec-12 polypeptide binds to cell surface ligands. Alternatively, the antibody can inhibit the biological activity of Siglec-12 polypeptide by inhibiting or preventing binding of Siglec-12 polypeptide to its cognate thus prevent inhibitory signaling of the ITIM domain. In one aspect, an agonistic antibody of the invention causes cross-linking of two or more Siglec-12 polypeptides thereby inducing a Siglec-12 biological activity.

The antibodies of the invention can be used in combination with other antibodies or therapeutics (including, e.g., soluble Siglec-12 polypeptide fragments). For example, anti-CD33 antibodies have shown both diagnostic and therapeutics uses in certain cell proliferative disorders. However, such anti-CD33 antibodies are not 100% efficacious. This may be due in part to the role of more than one molecule (e.g., more than one siglec molecule) playing a role in NK cell activation and inhibition as well as in cell-cell or cell-matrix adhesions. Accordingly, the anti-Siglec-12 polypeptide antibodies of the invention can increase the efficacy of the anti-CD33 antibodies or other therapeutics, when used in combination.

Compositions comprising an antibody that is directed against a Siglec-12 polypeptide or fragment thereof and a physiologically acceptable diluent, excipient, or carrier, are provided herein. Suitable components of such compositions are as described here and are similar to those described for compositions containing a Siglec-12 polypeptide or fragment thereof.

Also provided herein are conjugates comprising a detectable (e.g., diagnostic) or therapeutic agent, attached to the antibody. The conjugates find use in in vitro or in vivo procedures.

Polynucleotides The invention also provides Siglec-12 polynucleotides encoding Siglec-12 polypeptides and bioactive fragments thereof. A “polynucleotide” refers to a polymeric form of nucleotides of at least 10 bases in length. The nucleotides can be ribonucleotides, deoxyribonucleotides, or modified forms of either type of nucleotide. The term includes single and double stranded forms of DNA or RNA. DNA includes, for example, cDNA, genomic DNA, chemically synthesized DNA, DNA amplified by PCR, and combinations thereof. The polynucleotides of the invention include full-length genes and cDNA molecules as well as a combination of fragments thereof. The polynucleotides of the invention are preferentially derived from human sources, but the invention includes those derived from non-human species as well.

By “isolated polynucleotide” is meant a polynucleotide that is not immediately contiguous with both of the coding and/or non-coding sequences with which it is immediately contiguous (one on the 5′ end and one on the 3′ end) in the naturally occurring genome of the organism from which it is derived. The term therefore includes, for example, a recombinant polynucleotide molecule, which is incorporated into a vector, e.g., an expression vector; into an autonomously replicating plasmid or virus; or into the genomic DNA of a prokaryote or eukaryote, or which exists as a separate molecule (e.g., a cDNA) independent of other sequences.

A polynucleotide of the invention comprises (1) a sequence as set forth in SEQ ID NO:1; (2) sequences complementary to a sequence as set forth in SEQ ID NO:1; (3) fragments of SEQ ID NO:1 or their complements that specifically hybridize to the polynucleotide of (1) or (2) under moderate to highly stringent conditions, wherein the fragments are about 20 to 50 consecutive bases in length, 50 to 100 consecutive bases in length, 200 to 300 consecutive bases in length, and/or 500 to 1000 consecutive bases in length; and (4) sequences of (1), (2), or (3) wherein T can also be U. Also encompassed by the invention are homologues of a polynucleotide of the invention. These homologues can be identified in several ways, including isolation of genomic or cDNA molecules from a suitable source, or computer searches of available sequence databases. Oligonucleotides or polynucleotides corresponding to the amino acid sequences described herein can be used as probes or primers for the isolation of polynucleotide homologues or as query sequences for database searches. Degenerate oligonucleotide sequences can be obtained by “back-translation” from the amino acid sequences (e.g., a sequence of SEQ ID NO:2). The polymerase chain reaction (PCR) procedure can be employed to isolate and amplify a polynucleotide encoding a Siglec-12 polypeptide. Fragments of the polynucleotides of the invention are useful as probes and primers to identify or amplify related sequence or obtain full-length sequences of a Siglec-12 polynucleotide of the invention. The oligonucleotides can additionally contain recognition sites for restriction endonucleases, to facilitate insertion of the amplified combination of DNA fragments into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, Innis et al., eds., Academic Press, Inc. (1990).

The invention also includes polynucleotides and oligonucleotides that hybridize under reduced stringency conditions, more preferably moderately stringent conditions, and most preferably highly stringent conditions, to polynucleotides encoding Siglec-12 polypeptides described herein. The basic parameters affecting the choice of hybridization conditions and guidance for devising suitable conditions are set forth by Sambrook, J., E. F. Fritsch, and T. Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, F. M. Ausubel et al., eds., John Wiley & Sons, Inc., sections 2.10 and 6.3-6.4, incorporated herein by reference), and can be readily determined by those having ordinary skill in the art based on, for example, the length and/or base composition of the DNA. One way of achieving moderately stringent conditions involves the use of a prewashing solution containing 5× SSC, 0.5% SDS, 1.0 mM EDTA (pH 8.0), hybridization buffer of about 50% formamide, 6× SSC, and a hybridization temperature of about 55° C. (or other similar hybridization solutions, such as one containing about 50% formamide, with a hybridization temperature of about 42° C.), and washing conditions of about 60° C., in 0.5× SSC, 0.1% SDS. Generally, highly stringent conditions are defined as hybridization conditions as above, but with washing at approximately 68° C., 0.2× SSC, 0.1% SDS. SSPE (1× SSPE is 0.15M NaCl, 10 mM NaH₂PO₄, and 1.25 mM EDTA, pH 7.4) can be substituted for SSC (1× SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target polynucleotide of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 10° C. less than the melting temperature (Tₘ) of the hybrid, where Tm is determined according to the following equations. For hybrids less than 18 base pairs in length, Tₘ (° C.)=2(# of A+T bases)+4(# of G+C bases). For hybrids above 18 base pairs in length, Tₘ (° C.)=81.5+16.6(log10 [Na⁺])+0.41(% G+C)−(600/N), where N is the number of bases in the hybrid, and [Na⁺] is the concentration of sodium ions in the hybridization buffer ([Na⁺] for 1× SSC=0.165M). Typically each such hybridizing nucleic acid has a length that is at least 25% (more preferably at least 50%, or at least 60%, or at least 70%, and most preferably at least 80%) of the length of the nucleic acid of the invention to which it hybridizes, and has at least 60% sequence identity (more preferably at least 70%, at least 75%, at least 80%, at least 85%, at least 90%, at least 95%, at least 97.5%, or at least 99%, and most preferably at least 99.5%) with the nucleic acid of the invention to which it hybridizes.

Other embodiments of the invention include polynucleotides having sequences that encode discrete domains of a Siglec-12 polypeptide having a sequence of SEQ ID NO:2. Computer analysis predicts that the signal peptide of the Siglec-12 polypeptides is most likely to be cleaved after residue 13 of SEQ ID NO:2, though other possible cleavage sites include after amino acids 14, 15, or 16. These cleavage sites predict a mature Siglec-12 polypeptide comprising from about amino acid 14 to 686, from about amino acid 15 to 686, from about amino acid 16 to 686, or from about amino acid 17 to 686 of SEQ ID NO:2. The one or more, or a combination of the five Ig-like domains located from about amino acid 14 to 141, from about amino acid 142 to 235, from about amino acid 253 to 340, from about amino acid 357 to 443, and from about amino acid 444 to 538 of SEQ ID NO:2 are likely to be involved in cognate binding and transduction of extracellular signals to the ITIM domain(s). A transmembrane region is found at about amino acids 550 to 570, and a cytoplasmic domain from about amino acids 571 to 686. Thus, the invention provides polynucleotides encoding these discrete polypeptide fragments, as well as the polypeptide fragments comprising each domain separately or in various combinations. The invention provides polynucleotides comprising from about nucleotide 1 to 39, from about nucleotide 1 to 42, from about nucleotide 1 to 45, and from about nucleotide 1 to 48 of SEQ ID NO:1, which encode the signal peptides residing at amino acids 1-13, 1-14, 1-15, or 1-16 of SEQ ID NO:2, respectively; from about nucleotide 40 to 2058, from about nucleotide 43 to 2058, from about nucleotide 46 to 2058, or from about nucleotide 49 to 2058 of SEQ ID NO:1, which encode mature Siglec-12 polypeptides comprising amino acids 14-686, 15-686, 16-686, or 17-686 of SEQ ID NO:2, respectively; from about nucleotide 1648 to 1710 of SEQ ID NO:1, encoding a transmembrane region comprising amino acids 550-570 of SEQ ID NO:2 (which in some embodiments, is specifically excluded from a polynucleotide comprising an extracellular domain and/or intracellular domain); from about nucleotide 40 to 1647, from about nucleotide 43 to 1647, from about nucleotide 46 to 1647, or from about nucleotide 49 to 1647 of SEQ ID NO:1, encoding extracellular portions of a Siglec-12 polypeptide; and from about nucleotide 1711 to 2058 of SEQ ID NO:1, encoding a cytoplasmic domain comprising amino acids 571-686 of SEQ ID NO:2.

Polynucleotides of the invention may be used in developing treatments for any disorder mediated (directly or indirectly) by defective, or insufficient amounts of, a Siglec-12 gene corresponding to a polynucleotide of the invention. Disclosure, herein, of sequences corresponding to the polynucleotides of the invention permits the detection of defective genes, and the replacement thereof with normal genes. Defective genes may be detected in in vitro diagnostic assays, and by comparison of the polynucleotide sequences disclosed herein with that of a gene derived from a person suspected of harboring a defect in a Siglec-12 gene.

Other useful fragments of the disclosed polynucleotides include antisense or sense oligonucleotides comprising a single-stranded polynucleotide sequence (either RNA or DNA) capable of binding to target mRNA (sense) or DNA (antisense) sequences. Antisense or sense oligonucleotides, according to the invention, comprise fragments of the polynucleotide having a sequence as set forth in SEQ ID NO:1. Such a fragment generally comprises at least about 14 nucleotides, typically from about 14 to about 30 nucleotides. The ability to derive an antisense or a sense oligonucleotide, based upon a nucleic acid sequence encoding a given protein is described in, for example, Stein and Cohen (Cancer Res. 48:2659, 1988), and van der Krol et al. (BioTechniques 6:958, 1988).

Binding of antisense or sense oligonucleotides to target nucleic acid sequences results in the formation of duplexes that block or inhibit protein expression by one of several means, including enhanced degradation of the mRNA by RNAse H, inhibition of splicing, premature termination of transcription or translation, or by other means. The antisense oligonucleotides thus may be used to block expression of proteins. Antisense or sense oligonucleotides further comprise oligonucleotides having modified sugar-phosphodiester backbones (or other sugar linkages, such as those described in WO91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10448, and other moieties that increases affinity of the oligonucleotide for a target nucleic acid sequence, such as poly-(L)-lysine. Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid by any gene transfer method, including, for example, lipofection, CaPO₄-mediated DNA transfection, electroporation, or by using gene transfer vectors such as Epstein-Barr virus or adenovirus.

Sense or antisense oligonucleotides also may be introduced into a cell containing the target nucleic acid by formation of a conjugate with a ligand-binding molecule, as described in WO 91/04753. Suitable ligand binding molecules include, but are not limited to, cell surface receptors, growth factors, other cytokines, or other ligands that bind to cell surface receptors. Preferably, conjugation of the ligand-binding molecule does not substantially interfere with the ability of the ligand-binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid by formation of an oligonucleotide-lipid complex, as described in WO 90/10448. The sense or antisense oligonucleotide-lipid complex is preferably dissociated within the cell by an endogenous lipase.

In addition, “conservatively modified variants” applies to both polypeptides and polynucleotides. With respect to a particular polynucleotide, conservatively modified variants refer to codons in the polynucleotide which encode identical or essentially identical amino acids. Because of the degeneracy of the genetic code, a large number of functionally identical polynucleotides encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such variations are “silent variations,” which are one species of conservatively modified variations. Every polynucleotide sequence herein that encodes a polypeptide also describes every possible silent variation of the nucleic acid. One of skill will recognize that each codon in a polynucleotide (except AUG, which is ordinarily the only codon for methionine) can be modified to yield a functionally identical molecule. Accordingly, each silent variation of a nucleic acid that encodes a polypeptide is implicit in each described sequence.

The polynucleotides of the invention enable the construction of expression vectors comprising a polynucleotide encoding a Siglec-12 polypeptide or fragment thereof; host cells transfected or transformed with the expression vectors; isolated and purified biologically active polypeptides and bioactive fragments thereof; the use of the polynucleotides or oligonucleotides thereof as probes to identify nucleic acids encoding related siglec family proteins; the use of the polynucleotides or oligonucleotides thereof to correlate the location of genes encoding Siglec-12 polypeptides of the invention with chromosome regions associated with human diseases; the use of the polynucleotides, or oligonucleotides thereof, to identify genes associated with tumors, immune disorders, syndromes or other human conditions, as reagents for tissue-typing; the administration of the disclosed polypeptides or fragments thereof for the treatment of disorders characterized by a mutation in a gene encoding a Siglec-12 polypeptide or by an excess or a deficiency of a Siglec-12 polypeptide; the use of single-stranded sense or antisense oligonucleotides to inhibit expression of polynucleotides encoding a Siglec-12 polypeptide; the use of the disclosed polypeptides and soluble fragments thereof as competitive inhibitors of the binding of native Siglec-12 polypeptides to their ligands, cognates, or counter-structure binding partners; the use of Siglec-12 polypeptides and fragments thereof as unique molecular weight markers or as controls for peptide fragmentation and kits comprising these reagents; the use of Siglec-12 polypeptides and fragments thereof to generate antibodies; the use of such antibodies to purify Siglec-12 polypeptides; as affinity reagents for the separation of hematopoietic cells expressing the proteins; and the use of antibodies in the modulation of Siglec-12 polypeptide biological activity.

Expression, isolation and purification of the polypeptides and fragments of the invention may be accomplished by any suitable technique, including the utilization of expression systems such as those known in the art as well as those described herein.

In one embodiment, the invention provides an expression vector comprising a polynucleotide encoding a Siglec-12 polypeptide of the invention. The polynucleotide of the invention (e.g., a polynucleotide comprising a sequence as set forth in SEQ ID NO:1) may be operably inserted into, for example, a commercially available expression vector by recombinant techniques known in the art. Typically the polynucleotide will be inserted downstream (or 3′) of, and operably linked to, a control or regulatory sequence. As used herein, a “control sequence” or “regulatory sequence” are used interchangeably to include a promoter, enhancer-promoter combination, or other sequence that effects the expression or transcription of the downstream polynucleotide sequence. A promoter is a transcriptional regulatory element composed of a region of a DNA molecule typically within 100 nucleotide pairs in front of (upstream of) the point at which transcription starts. Another transcriptional regulatory element is an enhancer, which provides specificity in terms of time, location, and expression level. Unlike a promoter, an enhancer can function when located at variable distances from the transcription site, provided a promoter is present. An enhancer can also be located downstream of the transcription initiation site. Other regulatory sequences include transcription termination sequence, internal ribosome entry sites (IRES), and the like.

Typically, to bring a coding sequence under control of a promoter, it is necessary to position the translation initiation site of the translational reading frame of the peptide or polypeptide between one and about fifty nucleotides downstream (3′) of the promoter. Such regulatory elements include, but are not limited to, the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors, to name a few.

Expression vectors and methods for their construction are known to those skilled in the art (Ausubel et al., cited herein). Suitable vectors include plasmids, and viral vectors such as herpes viruses, retroviruses, canary poxviruses, adenoviruses and adeno-associated viruses, among others, and derivatives thereof.

A polynucleotide and regulatory sequences are “operably linked” when they are connected in such a way as to permit expression when the coding sequence (e.g., the Siglec-12 polypeptide coding sequence) of the polynucleotide is bound to the regulatory sequences, e.g., within an expression vector. An origin of replication that confers the ability to replicate in the desired host cells, and a selection gene (e.g., kan^{r}, amp^{r}) by which transformants are identified, are generally incorporated into the expression vector.

Expression vectors comprising a polynucleotide of the invention may be used to prepare the polypeptides or fragments of the invention encoded by the polynucleotide. A method for producing polypeptides comprises culturing host cells transformed or tranfected with a recombinant expression vector encoding the polypeptide, under conditions that promote expression of the polypeptide, then recovering the expressed polypeptides from the cells or from culture medium in which the host cell is grown. The procedure for purifying the expressed polypeptides will vary according to the type of host cells employed, and whether the polypeptide is membrane-bound or is a secreted soluble form of the polypeptide.

In addition, a sequence encoding an appropriate signal peptide (native or heterologous) can be incorporated into expression vectors. A DNA sequence for a signal peptide may be fused in frame to a polynucleotide sequence of the invention so that the polynucleotide is initially transcribed, and the mRNA translated, into a fusion protein comprising the signal peptide. Signal peptides may be employed that direct transmembrane proteins to the cell surface, or different signal peptides may be used that promote the secretion of a soluble form of the protein. Generally, the signal peptide is cleaved during maturation of the protein. A polynucleotide encoding a localization sequence, or signal sequence, can be ligated or fused at the 5′ terminus of a polynucleotide encoding a Siglec-12 polypeptide such that the signal peptide is located at the amino terminal end of the resulting fusion polynucleotide/polypeptide. In eukaryotes, the signal peptide functions to transport the fusion polypeptide across the endoplasmic reticulum. The secretory protein is then transported through the Golgi apparatus, into secretory vesicles and into the extracellular space or, preferably, the external environment. Signal peptides, which can be utilized according to the invention, include pre-pro peptides, which contain a proteolytic enzyme recognition site.

The localization sequence can be a nuclear localization sequence, an endoplasmic reticulum localization sequence, a peroxisome localization sequence, a mitochondrial localization sequence, or a localized protein. Localization sequences can be targeting sequences that are described, for example, in “Protein Targeting”, chapter 35 of Stryer, L., Biochemistry (4th ed.). W. H. Freeman, 1995. Some important localization sequences include those targeting the nucleus (e.g., KKKRK (SEQ ID NO:15)), mitochondrion (MLRTSSLFTRRVQPSLFRNILRLQST (SEQ ID NO:16)), endoplasmic reticulum (KDEL (SEQ ID NO:17)), peroxisome (SKF), prenylation or insertion into plasma membrane (CAAX (SEQ ID NO:18), CC, CXC, or CCXX (SEQ ID NO:19)), cytoplasmic side of plasma membrane (fusion to SNAP-25), or the Golgi apparatus (fusion to furin). Other examples of heterologous signal peptides that are functional in mammalian host cells include the signal sequence for interleukin-7 (IL-7) described in U.S. Pat. No. 4,965,195; the signal sequence for interleukin-2 receptor described in Cosman et al., Nature 312:768 (1984); the interleukin-4 receptor signal peptide described in EP 367,566; the type I interleukin-1 receptor signal peptide described in U.S. Pat. No. 4,968,607; and the type II interleukin-1 receptor signal peptide described in EP 460,846.

The skilled artisan will also recognize that the position(s) at which the signal peptide is cleaved may differ from that predicted by computer program, and may vary according to such factors as the type of host cells employed in expressing a recombinant polypeptide. A protein preparation may include a mixture of protein molecules having different N-terminal amino acids, resulting from cleavage of the signal peptide at more than one site. Particular embodiments of mature Siglec-12 polypeptides provided herein having a native signal sequence include, but are not limited to, polypeptides wherein the N-terminus amino acid is amino acid 14, 15, 16 or 17 of SEQ ID NO:2.

Suitable host cells for expression of polypeptides include prokaryotes (e.g., E. coli), yeast, plant cells, and insect or higher eukaryotic cells. Most typically, yeast or mammalian cells are used. Appropriate cloning and expression vectors for use with bacterial, fungal, yeast, and mammalian cellular hosts are described, for example, in Pouwels et al. Cloning Vectors: A Laboratory Manual, Elsevier, N.Y., (1985). Cell-free translation systems could also be employed to produce polypeptides using RNAs derived from DNA constructs disclosed herein.

Suitable prokaryotic host cells for transformation may be gram-negative or gram-positive, and include, for example, E. coli, Bacillus subtilis, Salmonella typhimurium, and various other species within the genera Pseudomonas, Streptomyces, and Staphylococcus. In a prokaryotic host cell, such as E. coli, a polypeptide may include an N-terminal methionine (met) residue to facilitate expression of the recombinant polypeptide in the prokaryotic host cell. The N-terminal Met may be cleaved from the expressed recombinant polypeptide.

Expression vectors for use in prokaryotic host cells generally comprise one or more phenotypic selectable marker genes, which may include, for example, a gene encoding a protein that confers antibiotic resistance or that supplies an autotrophic requirement. Useful prokaryotic expression vectors include those derived from commercially available plasmids such as the cloning vector pBR322 (ATCC 37017), with ampicillin and tetracycline resistance genes. Other suitable vectors include, pKK223-3 (Pharmacia Fine Chemicals, Uppsala, Sweden) and pGEM1 (Promega Biotec, Madison, Wis., USA). An appropriate promoter and a polynucleotide sequence encoding the desired polypeptide may be inserted into the vector.

Promoter sequences commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980) and tac promoter (Maniatis et al., Molecular Cloning: A Laboratory Manual, first ed., Cold Spring Harbor Laboratory, p. 412, 1982). A particularly useful prokaryotic host cell expression system employs a phage λPL promoter and a cI857ts thermolabile repressor sequence. Plasmid vectors available from the American Type Culture Collection which incorporate derivatives of the λPL promoter include plasmid pHUB2 (resident in E. coli strain JMB9, ATCC 37092) and pPLc28 (resident in E. coli RR1, ATCC 53082).

Alternatively, the polypeptides may be expressed in yeast host cells, such as from the Saccharomyces genus (e.g., S. cerevisiae). Alternatively, Pichia, Kluyveromyces, or other yeast genera may be employed. Yeast vectors will often contain an origin of replication sequence from a 2mu yeast plasmid, an autonomously replicating sequence (ARS), a promoter region, sequences for polyadenylation, sequences for transcription termination, and a selectable marker gene. Suitable promoter sequences include those derived from the yeast metallothionein or 3-phosphoglycerate kinase genes (Hitzeman et al., J. Biol. Chem. 255:2073, 1980) or other genes encoding glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phospho-glucose isomerase, and glucokinase. Other suitable vectors and promoters for use in yeast expression are known in the art (e.g., see in Hitzeman, EPA-73,657; Russell et al., J. Biol. Chem. 258:2674, 1982; and Beier et al., Nature 300:724, 1982).

The yeast α-factor leader sequence may be employed to direct secretion of the polypeptide, and often is inserted between the promoter sequence and the structural gene sequence (e.g., Kurjan et al., Cell 30:933, 1982 and Bitter et al., Proc. Natl. Acad. Sci. USA 81:5330, 1984).

Yeast transformation protocols are known to those of skill in the art, including a protocol involving selection for Trp⁺ transformants in a medium containing yeast nitrogen base, casamino acids, glucose, 10 mg/ml adenine and 20 mg/ml uracil (e.g., Hinnen et al., Proc. Natl. Acad. Sci. USA 75:1929, 1978). In other protocols, yeast cells transformed by vectors containing an ADH2 promoter sequence may be grown in a “rich” medium. An example of a rich medium is one consisting of 1% yeast extract, 2% peptone, and 1% glucose supplemented with 80 mg/ml adenine and 80 mg/ml uracil. Derepression of the ADH2 promoter occurs when glucose is exhausted from the medium.

Mammalian or insect host cell culture systems also may be employed to express recombinant polypeptides, such as the bacculovirus systems reviewed by Luckow and Summers, Bio/Technology 6:47 (1988). Established cell lines of mammalian origin also may be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV1/EBNA cell line derived from the African green monkey kidney cell line CV1 (ATCC CCL 70) as described by McMahan et al. (EMBO J. 10: 2821, 1991).

Established methods for introducing polynucleotides into mammalian cells have been described (Kaufman, R. J., Large Scale Mammalian Cell Culture, 1990, pp. 15-69). Additional protocols using commercially available reagents, such as Lipofectamine lipid reagent (Gibco/BRL) or Lipofectamine-Plus lipid reagent, can be used to transfect cells (Felgner et al., Proc. Natl. Acad. Sci. USA 84:7413-7417, 1987). In addition, electroporation can be used to transfect mammalian cells using conventional procedures, such as those in Sambrook et al., 1989. Selection of stable transformants can be performed using methods known in the art, such as, for example, resistance to cytotoxic drugs. Kaufman et al., Meth. in Enzymology 185:487-511, 1990, describes several selection schemes, such as dihydrofolate reductase (DHFR) resistance. A suitable host strain for DHFR selection is CHO strain DX-B11, which is deficient in DHFR (Urlaub and Chasin, Proc. Natl. Acad. Sci. USA 77:4216-4220, 1980). A plasmid expressing the DHFR cDNA can be introduced into strain DX-B11, and only cells that contain the plasmid can grow in the appropriate selective media. Other examples of selectable markers include genes conferring resistance to antibiotics, such as G418 and hygromycin B, which permit selection of cells harboring the vector on the basis of resistance to these agents.

Transcriptional and translational control sequences for mammalian host cell expression vectors can be excised from viral genomes. Commonly used promoter sequences and enhancer sequences are derived from polyoma virus, adenovirus 2, simian virus 40 (SV40), and human cytomegalovirus. Polynucleotide sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al., Nature 273:113, 1978; Kaufman, Meth. in Enzymology, 1990). Smaller or larger SV40 fragments can also be used, provided the approximately 250 bp sequence extending from the Hind III site toward the Bgl I site located in the SV40 viral origin of replication site is included.

Additional control sequences shown to improve expression of heterologous genes from mammalian expression vectors include such elements as the expression augmenting sequence element (EASE) derived from CHO cells (Morris et al., Animal Cell Technology, 1997, pp. 529-534 and PCT Application WO 97/25420) and the tripartite leader (TPL) and VA gene RNAs from Adenovirus 2 (Gingeras et al., J. Biol. Chem. 257:13475-13491, 1982). The internal ribosome entry site (IRES) sequences of viral origin allows dicistronic mRNAs to be translated efficiently (Oh and Sarnow, Current Opinion in Genetics and Development 3:295-300, 1993; Ramesh et al., Nucleic Acids Research 24:2697-2700, 1996). Expression of a heterologous cDNA as part of a dicistronic mRNA followed by the gene for a selectable marker (e.g. DHFR) has been shown to improve transfectability of the host and expression of the heterologous polynucleotides (Kaufman, Meth. in Enzymology, 1990). Exemplary expression vectors that employ dicistronic mRNAs are pTR-DC/GFP described by Mosser et al., Biotechniques 22:150-161, 1997, and p2A5I described by Morris et al., Animal Cell Technology, 1997, pp. 529-534.

A useful high expression vector, pCAVNOT, has been described by Mosley et al., Cell 59:335-348, 1989. Other expression vectors for use in mammalian host cells can be constructed as disclosed by Okayama and Berg (Mol. Cell. Biol. 3:280, 1983). A useful system for stable high level expression of mammalian cDNAs in C127 murine mammary epithelial cells can be constructed substantially as described by Cosman et al. (Mol. Immunol. 23:935, 1986). A useful high expression vector, PMLSV N1/N4, described by Cosman et al., Nature 312:768, 1984, has been deposited as ATCC 39890. Additional useful mammalian expression vectors are described in EP-A-0367566, and in WO 91/18982, incorporated by reference herein. In yet another alternative, the vectors can be derived from retroviruses.

Additional useful expression vectors, pFLAG® and pDC311, can also be used. FLAG® technology is centered on the fusion of a low molecular weight (1 kD), hydrophilic, FLAG® marker peptide to the N-terminus of a recombinant protein expressed by pFLAG® expression vectors. pDC311 is another specialized vector used for expressing proteins in CHO cells. pDC311 is characterized by a bicistronic sequence containing the gene of interest and a dihydrofolate reductase (DHFR) gene with an internal ribosome binding site for DHFR translation, an expression augmenting sequence element (EASE), the human CMV promoter, a tripartite leader sequence, and a polyadenylation site.

Activity Assays

The purified polypeptides of the invention (including proteins, polypeptides, fragments, variants, oligomers, and other forms) may be tested for the ability to bind a Siglec-12 polypeptide-binding partner, such as a sialic acid-containing protein (e.g., an α2-8 sialic acid moiety), in any suitable assay, such as a conventional binding assay (see, e.g., Patel et al., J. Biol. Chem. 274:22729-22738, 1999; Angata and Varki, J. Biol. Chem. 275:22127-22135, 2000; and Angata and Varki, Glycobiology, 10:431438, 2000). In another aspect, a polypeptide may be labeled with a detectable reagent (e.g., a radionuclide, chromophore, enzyme that catalyzes a calorimetric or fluorometric reaction, and the like), and then contacted with cells expressing a Siglec-12 polypeptide binding partner surface protein. The cells are washed to remove unbound labeled polypeptide, and the presence of cell-bound label is determined by a suitable technique. For example, a recombinant expression vector is constructed containing a polynucleotide encoding a Siglec-12 polypeptide (or bioactive fragment thereof) fused to an Fc region according to methods well known in the art. Upon expression the polynucleotide may encode, for example, a soluble Siglec-12 polypeptide comprising the extracellular portions of the Siglec-12 polypeptide, or may encode the extracellular domain and a cytoplasmic domain with the transmembrane region removed. Host cells are transfected with the recombinant expression vector comprising a polynucleotide of the invention. The transfected cells are cultured. After culturing, medium containing a Siglec-12 polypeptide or other soluble polypeptide of the invention is collected from the transfected cells and the amount of the polypeptide is quantified using standard methods.

Cells expressing a binding partner (e.g., a sialic acid containing molecule) are cultured as above, and washed with BM-NFDM, which is binding medium (RPMI 1640 containing 25 mg/ml bovine serum albumin, 2 mg/ml sodium azide, 20 mM Hepes pH 7.2) to which 50 mg/ml nonfat dry milk has been added. The cells then are incubated, for example, with various concentrations of a soluble Siglec-12-polypeptide/Fc fusion polypeptide made as set forth above. Cells are washed and incubated with a constant saturating concentration of a ¹²⁵I-mouse anti-human IgG in binding medium, with gentle agitation for 1 hour at 37° C. After extensive washing, cells are released via trypsinization.

The mouse anti-human IgG employed above is directed against the Fc region of human IgG and can be obtained from Jackson Immunoresearch Laboratories, Inc., West Grove, Pa. The antibody is radioiodinated using the standard chloramine-T method. The antibody will bind to the Fc portion of any polypeptide/Fc protein that has bound to the cells. In all assays, non-specific binding of ¹²⁵I-antibody is assayed in the absence of the Fc fusion protein, as well as in the presence of the Fc fusion protein and a 200-fold molar excess of unlabeled mouse anti-human IgG antibody.

Cell-bound ¹²⁵I-antibody is quantified on a Packard Autogamma counter. Affinity calculations (Scatchard, Ann. N.Y. Acad. Sci. 51:660, 1949) are generated on RS/1 (BBN Software, Boston, Mass.) run on a Microvax computer.

Another type of suitable binding assay is a competitive binding assay. To illustrate, biological activity of a variant may be determined by assaying for the variant's ability to compete with the native proteins for binding to its binding partner.

Competitive binding assays can be performed by conventional methodology. Reagents that may be employed in competitive binding assays include a radiolabeled soluble Siglec-12 polypeptide or intact cells expressing a Siglec-12 polypeptide (endogenous or recombinant) on the cell surface. For example, a radiolabeled bioactive fragment of a Siglec-12 polypeptide can be used to compete with a soluble variant for binding to a cell surface-binding partner. Instead of intact cells, one could substitute a bioactive fragment of a Siglec-12 polypeptide/Fc fusion protein bound to a solid phase through the interaction of Protein A or Protein G (on the solid phase) with the Fc moiety. Chromatography columns that contain Protein A and Protein G include those available from Pharmacia Biotech, Inc., Piscataway, N.J.

Another type of competitive binding assay utilizes a radiolabeled soluble bioactive fragment of a Siglec-12 polypeptide, such as a soluble bioactive fragment/Fc fusion protein, and intact cells expressing Siglec-12 binding partners. Qualitative results can be obtained by competitive autoradiographic plate binding assays, while Scatchard plots (Scatchard, Ann. N.Y. Acad. Sci. 51:660, 1949) may be utilized to generate quantitative results.

Another type of assay is a rosetting assay. For example COS-7 are transfected with an expression vector containing Siglec-12 or a fragment thereof. The cells are then contacted with normal red blood cells (RBCs) and incubated for approximately 30 minutes followed by removal of nonadherent RBCs by gentle washes. The number of RBCs bound to each COS-7 cell is then quantitated and is indicative of the number of rosettes formed. Rosette formation is indicative of an interaction of the Siglec-12 or fragment thereof with its cognate. Modification of the above include the use of antibodies to siglec-12 to prevent rosette formation or use of soluble fragments of siglec-12 to prevent rosette formation.

Diagnostic Assays

The polynucleotides and polypeptides provided herein are useful as diagnostic reagents. Samples for diagnostic reagents may be obtained from a subject's tissues, for example, throat swab, blood, serum, urine, saliva, cerebrospinal fluid, feces, tissue biopsy, and so on. Similar samples are taken from normal individuals (from persons not suffering from the disorder in question), and these normal or standard samples may provide a basis for comparison. Purified reagents (e.g., Siglec-12 polynucleotides, polypeptides, and antibodies) may be used as standards for the diagnostic assays. In some embodiments, fragments of the polynucleotides of the invention are used as probes for Northern or Southern blots or as PCR primers to detect mutated forms of a Siglec-12 polypeptide encoded by the target nucleic acid.

Conditions that may be diagnosed include those characterized by an excess or deficiency of a Siglec-12 polypeptide, or that are characterized by a mutated form of such a polypeptide. Such conditions include, but are not limited to, absence of the polypeptide in a cell that requires its expression, altered enzymatic activity, altered signaling ability, overexpression or underexpression.

Particular conditions that may be diagnosed using these assays include, but are not limited to: rheumatologic diseases (e.g., rheumatoid arthritis, psoriatic arthritis, seronegative spondyloarthropathies), inflammatory conditions, bone marrow or solid organ transplantation, graft-versus-host disease, allergies (e.g., asthma, allergic rhinitis), neurologic disorders (e.g., Alzheimer's, Parkinson's, dementia, brain cancer, Bell's palsy, post-herpetic neuralgia), cell proliferative disorders including neoplasms or cancer (e.g., lymphoma, B-cell, T-cell and myeloid cell leukemias), infections (e.g., bacterial, parasitic, protozoal and viral infections, including AIDS), chemotherapy or radiation-induced toxicity, cachexia, cardiovascular disorders (e.g., congestive heart failure, myocardial infarction, ischemia/reperfusion injury, arteritis, stroke), gastrointestinal disorders (e.g., inflammatory bowel disease, Crohn's disease, celiac disease), diabetes mellitus, skin diseases (e.g., psoriasis, scleroderma, dermatomyositis), hematologic disorders (e.g., myelodysplastic syndromes, acquired or Fanconi's aplastic anemia), septic shock, liver diseases (e.g., viral hepatitis or alcohol-associated), bone disorders (e.g., osteoporosis, osteopetrosis).

In some embodiments of the invention, the condition being diagnosed is a hematologic disorder, and the tissue sample is blood or a lymph node biopsy.

Screening for Modulators of Siglec-12 Polypeptides and Polynucleotides

The Siglec-12 polypeptides and polynucleotides disclosed herein find use in screening assays for identifying agents that modulate the expression or activity of the polynucleotides and polypeptides of the invention, respectively. Once identified, agents that modulate expression or activity of a Siglec-12 polynucleotide or polypeptide may be administered, for example, to suppress siglec expression in conditions characterized by overproduction of these or other siglecs. Similarly, agents that stimulate the biological activity or expression of a Siglec-12 polypeptide in cultured cells or in subjects may be administered to stimulate activity or expression where a condition is characterized by a deficiency of the normal endogenous activator of a siglec of the invention.

Methods to identify an agent that modulates the activity or expression of a Siglec-12 polypeptide can be carried out using the teachings provided herein. For example, to identify a test agent that modulates Siglec-12 polypeptide activity the test agent is contacted with a sample containing a Siglec-12 polypeptide of the invention. The sample is then assayed to measure Siglec-12 polypeptide activity and the Siglec-12 polypeptide activity in the presence of the test agent is compared to the activity present in a standard (i.e., a control) sample. A sample can be, for example, a cell-free sample, a cell-containing sample (e.g., a cell culture), or a tissue sample (e.g., a tissue sample obtained or derived from a subject). A standard sample includes, for example, the sample prior to contact with the test agent or a sample that represents normal activity. Activity can be measured using any of the assay methods identified herein (e.g., competitive binding assays and the like). A change in activity compared to a control or standard sample is indicative of an agent that modulates (e.g., increases or decreases) activity.

Similarly, the invention provides a method for identifying an agent that modulates expression of a Siglec-12 polypeptide. Such methods include, for example, contacting a sample comprising a polynucleotide of the invention with a test agent and measuring expression of the polynucleotide compared to a standard or control sample. The level of expression can be determined by methods know in the art, including detecting protein (e.g., by Western Blot), or by detecting the amount of mRNA transcribed (e.g., by PCR). As above, the sample can be a cellular sample, a tissue sample, and the like. A change in expression compared to a control or standard sample is indicative of an agent that modulates (e.g., increases or decreases) expression.

A test agent can include, for example, a protein, a peptide, a peptidomimetic, an antibody, a small molecule, or a polynucleotide (e.g., an antisense or ribozyme). An example of a test agent is a ligand that binds specifically with a Siglec-12 polypeptide, or other molecules capable of forming functional heteromers with the Siglec-12 polypeptide.

Cells used for these screening assays may include, for example, cells that naturally express a Siglec-12 polypeptide, such as glial cells, T-cells, myeloid cells, macrophages, microglial cells, and other hematopoietic cells, or any convenient cell type that has been transformed or transfected with a heterologous nucleic acid that directs the expression of a Siglec-12 polypeptide.

In other assays, cells expressing a bioactive fragment of a Siglec-12 polypeptide (e.g., a soluble form) may be cultured with the test molecule to determine whether the molecule has the capacity to modulate the amount of the bioactive fragment produced by the cells. The amount of bioactive fragment produced may be measured by any suitable method, including enzyme-linked immunosorbent assay (ELISA), dot blot employing an antibody that binds the bioactive fragment, or a solid phase binding assay.

Methods of Therapy

This invention provides compounds, compositions, and methods for treating a subject, such as a mammalian subject, and typically a human subject, who is suffering from a medical disorder, and in particular a Siglec-12 polypeptide-mediated disorder. Such Siglec-12 polypeptide-mediated disorders include conditions caused (directly or indirectly) or exacerbated by binding between a Siglec-12 polypeptide and a binding partner. For purposes of this disclosure, the terms “illness,” “disease,” “medical condition,” “abnormal condition” and the like are used interchangeably with the term “medical disorder.” The terms “treat”, “treating”, and “treatment” used herein includes curative, preventative (e.g. prophylactic) and palliative or ameliorative treatment.

The polypeptides and polynucleotides of the invention may be administered therapeutically to a mammalian subject (e.g., bovine, equine, feline, canine, porcine, primates), preferably a human subject, having a disorder involving a malfunctioning Siglec-12 gene or polypeptide, including an excess or a deficiency of such a polypeptide, or expression of a deleterious mutant form of the polypeptide. Such disorders include conditions caused (directly or indirectly) or exacerbated by such forms of the polypeptides.

For these therapeutic methods, agents that modulate activity or expression of a Siglec-12 polypeptide or polynucleotide, respectively, may be employed. Such modulating agents are identified by screening, such as by employing the screening methods disclosed herein. Antibodies that bind specifically with the Siglec-12 polypeptide or its ligand may modulate the biological activity of the Siglec-12 polypeptide.

Disorders and diseases treatable by the methods and compositions of the invention include, but are not limited to: rheumatologic disorders (e.g., rheumatoid arthritis, psoriatic arthritis, seronegative spondyloarthropathies), bone marrow or solid organ transplant, graft-versus-host reaction, inflammatory conditions, autoimmune disorders (e.g., systemic lupus erythematosus, Hashimoto's thyroiditis, Sjogren's syndrome), allergies (e.g., asthma, allergic rhinitis), neurologic disorders (e.g., Alzheimer's, Parkinson's, dementia, brain cancer, Bell's palsy, post-herpetic neuralgia), cancers (e.g., lymphoma, B-cell, T-cell and myeloid cell leukemias), infections (e.g., bacterial, parasitic, protozoal and viral infections, including AIDS), chemotherapy or radiation-induced toxicity, cachexia, cardiovascular disorders (e.g., congestive heart failure, myocardial infarction, ischemia/reperfusion injury, arteritis, stroke), diabetes mellitus, skin diseases (e.g., psoriasis, scleroderma, dermatomyositis), hematologic disorders (e.g., myelodysplastic syndromes, acquired or Fanconi's aplastic anemia), septic shock, liver diseases (e.g., viral hepatitis or alcohol-associated), bone disorders (e.g., osteoporosis, osteopetrosis).

For treating the above disorders, the therapeutic agent, may be administered in an amount effective to measurably reduce one or more signs or symptoms of the disorder being treated. In addition, such disorders may be treated by administration in vivo or ex vivo of a vector or liposome that delivers a non-defective form of the malfunctioning gene to the cell type in which the malfunction is present.

Therapeutic compositions may comprise a substantially purified Siglec-12 polypeptide in any form described herein, such as a native polypeptide, a variant, a derivative, an oligomer, and a bioactive fragment. The composition may comprise a soluble polypeptide or an oligomer comprising s soluble Siglec-12 polypeptide. In another embodiment, a composition comprises an antibody directed against at least one Siglec-12 polypeptide epitope. The antibody may be coupled to a toxin, radioisotope or other therapeutic agents and used to target the therapeutic agent to a cell expressing a Siglec-12 polypeptide.

Combination therapies also are envisioned, in which another pharmacologically active agent is co-administered with a therapeutic agent of the invention. Other agents suitable for co-administration include but are not limited to cytokines, lymphokines, chemokines, chemotherapy agents, anti-inflammatories, DMARDs, or any other compound effective in treating the target disease or disorder.

Pharmaceutical compositions of the invention furthermore may comprise other components such as a physiologically acceptable diluent, carrier, or excipient, and are formulated according to known methods. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa.

In addition, such compositions can be complexed with polyethylene glycol (PEG), metal ions, or incorporated into polymeric compounds such as polyacetic acid, polyglycolic acid, hydrogels, dextran, and the like, or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance, and are thus chosen according to the intended application.

The compositions of the invention can be administered in any suitable manner, e.g., orally, topically, parenterally, or by inhalation. The term “parenteral” includes injection, e.g., by subcutaneous, intravenous, or intramuscular routes, also including localized administration, e.g., at a site of disease or injury. Sustained release from implants is also contemplated. Suitable dosages will vary, depending upon such factors as the nature of the disorder to be treated, the patient's body weight, age, and general condition, and the route of administration. Preliminary doses can be determined according to animal tests, and the scaling of dosages for human administration is performed according to art-accepted practices.

The dose, route of administration, frequency of administration and duration of an effective regimen of treatment will vary, depending factors such as the particular condition being treated, the severity of the condition, the age of the subject, and the like, and may be adjusted accordingly by the subject's physician.

In one method of treatment, the active agent is a polypeptide, and is administered by injection one to three times a week at a dose ranging from 0.1-100 mg/kg, or more preferably at a dose of 0.4-50 mg/kg. Treatment is continued until a measurable improvement in the subject's condition has been ascertained, which in most cases will require at least two to eight weeks or more of treatment. Maintenance doses may be administered thereafter, and treatment may be resumed if evidence of disease should reappear. Suitable regimens for other routes of administration may be determined according to methods known in the art. Similarly, suitable regimens for administering antibodies, small molecules, antisense or gene therapy reagents may be determined according to methods known in the art.

Included within the scope of the invention are pharmacologically acceptable compositions comprising the aforedescribed therapeutic agents, including compositions suitable for administration by each of the aforedesribed routes. Such compositions are formulated in accord with standard practices.

The following examples are provided to further illustrate particular embodiments of the invention, and are not to be construed as limiting the scope of the invention.

### EXAMPLES

### Example 1

### Identification of a Siglec-Like Polypeptide

Prior siglec nucleic acid sequences have been localized to chromosome 19 of the human genome. A TBLASTN comparison of a known set of siglec polypeptide sequences to all six possible reading frames of a genomic sequence with accession number AC011452 (corresponding to chromosome 19) detected an initial putative novel siglec-homologue open reading frame (ORF). The putative Siglec-12 polypeptide-coding region was assembled electronically from the predicted genomic sequence identified and then aligned with other siglec coding regions to identify regions of lower sequence identity in the putative Siglec-12 sequence. One such region of low identity (corresponding to nucleotides 1651 to 1941 of SEQ ID NO:1) was chosen to prepare an oligonucleotide template (by PCR amplification) as a hybridization probe. A subsequence of the PCR product (corresponding to nucleotides 1651 to 1769 of SEQ ID NO:1) was radiolabeled and used to screen a human spleen cDNA library using standard conditions of moderate stringency. Several positive cDNA clones were identified and isolated, and their inserts prepared for DNA sequence analysis. Sequencing was carried out using standard techniques.

### Example 2

### Analysis of Siglec-Like Polypeptide

An analysis of the Siglec-12 polypeptide sequences predicted from the ORF demonstrates that SEQ ID NO:2 contains a predicted signal peptide, five Ig domains, a transmembrane domain and a cytoplasmic domain having homology to the siglec family of proteins. In addition, a number of conserved cysteine residues were identified (see FIG. 1). Several distinct regions can be discerned within the Siglec-12 polypeptides of the invention. A signal peptide is present in the Siglec-12 polypeptide. The signal peptide present in the full-length polypeptide of the invention is predicted to include from about amino acid 1 to 13 of SEQ ID NO:2. The signal peptide cleavage site for Siglec-12 polypeptide was predicted using a computer algorithm. However, one of skill in the art will recognize that the cleavage site of the signal sequence may vary depending upon a number of factors including the organism in which the polypeptide is expressed. Accordingly, the N-terminus of a mature form of a Siglec-12 polypeptide of the invention may vary by about 2 to 5 amino acids. Thus, a mature form of the Siglec-12 polypeptide of the invention may include at its N-terminus amino acids 9, 10, 11, 12, 13, 1, 15, 16, 17, 18, 19, or 20 of SEQ ID NO:2. Accordingly, the mature form comprises a sequence of about amino acid 9, 10, 11, 12, 13, 1, 15, 16, 17, 18, 19, or 20 to about amino acid 686 (or, in the case of a soluble polypeptide, 549) of SEQ ID NO:2. The extracellular regions of the Siglec-12 polypeptides are located at about amino acids 14 to 549 of SEQ ID NO:2. The Ig-like domain assignments, as well as those for the transmembrane and cytoplasmic domains are based upon computer algorithms, on previous reports (Foussias et al., Genomics 67:171-178, 2000; Foussias et al., Biochem Biophys. Res. Comm. 278:775-781, 2000; Floyd et al., J. Biol. Chem. 275:861-866, 2000) and the one domain-one exon rule (Willams and Barclay, Annu. Rev. Immunol. 6:381-405, 1988). The extracellular region of Siglec-12 polypeptide putatively contains five Ig-like domains located at about amino acids 14-141, 142-235, 253-340, 357-443, and 444-538 of SEQ ID NO:2. The transmembrane regions for these polypeptides are located at about amino acids 550 to 570 of SEQ ID NO:2. The intracellular/cytoplasmic regions are located at amino acids 571 to 686 of SEQ ID NO:2. The cytoplasmic portion of the Siglec-12 polypeptide contains a putative ITIM motif, as well as a second sequence that is a modified ITIM motif or a putative signaling lymphocyte activation molecule (SLAM) motif The first of these has the sequence LHYASL (SEQ ID NO:3), and corresponds to amino acids 630 to 635 of SEQ ID NO:2. The second motif sequence is TEYSEI (SEQ ID NO:4), corresponding to amino acids 654 to 659 of SEQ ID NO:2. This second motif has homology to a sequence (TxYxx(IV)) recently found in the signaling lymphocyte activation molecule (SLAM) that is responsible for the binding of SLAM-associated protein (SAP) (Coffey et al., Nat. Genet. 20:129-135, 1998; Foussias et al., Genomics 67:171-178, 2000). Alternatively, the second motif may represent a functional variant of the ITIM motif. FIG. 1 shows the relative domains and conserved residues of Siglec-12 polypeptide indicative of a siglec polypeptide. Conserved cysteine residues are highlighted. The signal sequence and the transmembrane sequence are underlined. The putative ITIM and SLAM sequences are highlighted. The first Ig domain is in bold, the second Ig domain is italicized, the third Ig domain is in reverse text, the fourth Ig domain is double underlined and the fifth Ig domain is dotted-underlined.

Variants of the Siglec-12 polypeptide sequences can be identified based upon the sequences provided herein. Variants are provided herein and are included within the scope of the invention. Amino acid substitutions and other alterations (deletions, insertions, and the like) to Siglec-12 polypeptides are predicted to be more likely to alter or disrupt Siglec-12 polypeptide activities if they result in changes to the conserved residues of the amino acid sequences as shown in FIG. 1, and particularly if those changes do not substitute an amino acid of similar structure (such as substitution of any one of the aliphatic residues—Ala, Gly, Leu, Ile, or Val—for another aliphatic residue). Conversely, if a change is made to a Siglec-12 polypeptide resulting in substitution of the residue at that position in the alignment from one of the other siglec polypeptide sequences, it is less likely that such an alteration will affect the function of the altered Siglec-12 polypeptide.

Intron/Exon boudaries for the Siglec-12 molecule were predicted as set forth in Table 1.

TABLE 1

| | Amino | | | |
|---|---|---|---|---|
| Exon Number | acid seq. | Junction | Codon | Intron size |
| Exon 1 | 1-13 | within Gly-14 | G/GG | 70 bp |
| Exon 2 | 15-141 | within Ala-142 | G/CC | 130 bp |
| Exon 3 | 143-236 | within Tyr-137 | T/AT | 316 bp |
| Exon 4 | 238-252 | within Ala-253 | G/GC | 149 bp |
| Exon 5 | 254-340 | within Tyr-341 | T/AT | 282 bp |
| Exon 6 | 342-356 | within Val-357 | G/TC | 129 bp |
| Exon 7 | 358-442 | within Tyr-443 | T/AC | 72 bp |
| Exon 8 | 444-538 | within Gly-539 | G/GG | 5888 bp |
| Exon 9 | 540-570 | within Arg-571 | AG/G | 325 bp |
| Exon 10 | 572-598 | following Gln-598 | CAG/ | 1655 bp |
| Exon 11 | 599-end | | | |

### Example 3

### Monoclonal Antibodies that Bind Siglec-12 Polypeptides

Substantially purified Siglec-12 polypeptides or fragments thereof can be used to generate monoclonal antibodies immunoreactive therewith, using conventional techniques such as those described in U.S. Pat. No. 4,411,993. Briefly, mice are immunized with a Siglec-12 polypeptide immunogen emulsified in complete Freund's adjuvant, and injected in amounts ranging from 10-100 μg subcutaneously or intraperitoneally. Ten to twelve days later, the immunized animals are boosted with additional Siglec-12 polypeptide, or fragment thereof, emulsified in incomplete Freund's adjuvant. Mice are periodically boosted thereafter on a weekly to bi-weekly immunization schedule. Serum samples are periodically taken by retro-orbital bleeding or tail-tip excision to test for Siglec-12 antibodies by dot blot assay, ELISA (Enzyme-Linked Immunosorbent Assay), or inhibition of binding of a Siglec-12 polypeptide to a Siglec-12 polypeptide binding partner.

Following detection of an appropriate antibody titer, positive animals are provided one last intravenous injection of a Siglec-12 polypeptide or fragment in saline. Three to four days later, the animals are sacrificed, spleen cells harvested, and spleen cells are fused to a murine myeloma cell line, e.g., NS1 or preferably P3x63Ag8.653 (ATCC CRL 1580). Fusions generate hybridoma cells, which are plated in multiple microtiter plates in a HAT (hypoxanthine, aminopterin and thymidine) selective medium to inhibit proliferation of non-fused cells, myeloma hybrids, and spleen cell hybrids.

The hybridoma cells are screened by ELISA for reactivity against a substantially pure Siglec-12 polypeptide by adaptations of the techniques disclosed in Engvall et al., (Immunochem. 8:871, 1971) and in U.S. Pat. No. 4,703,004. A preferred screening technique is the antibody capture technique described in Beckmann et al., (J. Immunol. 144:4212, 1990). Positive hybridoma cells can be injected intraperitoneally into syngeneic BALB/c mice to produce ascites containing high concentrations of anti-Siglec-12 polypeptide monoclonal antibody. Alternatively, hybridoma cells can be grown in vitro in flasks or roller bottles by various techniques. Monoclonal antibodies produced in mouse ascites can be purified by ammonium sulfate precipitation, followed by gel exclusion chromatography. Alternatively, affinity chromatography based upon binding of antibody to Polypeptide A or Polypeptide G can also be used, as can affinity chromatography based upon binding to siglec polypeptide.

### EXAMPLE 4

### Chromosome Mapping

Chromosome mapping can be carried out in, for example, one of the following two methods. The gene corresponding to a Siglec-12 polypeptide is mapped using PCR-based mapping strategies. Initial human chromosomal assignments are made using Siglec-12-specific PCR primers and a BIOS Somatic Cell Hybrid PCRable DNA kit from BIOS Laboratories (New Haven, Conn.), following the manufacturer's instructions. More detailed mapping is performed using a Genebridge 4 Radiation Hybrid Panel (Research Genetics, Huntsville, Ala.; described in Walter, M A et al., Nature Genetics 7:22-28, 1994). Data from this analysis is then submitted electronically to the MIT Radiation Hybrid Mapper (URL: http://www-genome.wi.mit.edu/cgi-bin/contig/rhmapper.p1) following the instructions contained therein. This analysis yields specific genetic marker names which, when submitted electronically to the NCBI Genemap browser (www-ncbi.n1m.nih.gov80/cgi-bin/enterez/hum_srch?chr=hum_chr.ing&guery), yield the specific chromosome interval.

Alternatively, database analysis can yield information on the location of the polynucleotide sequence encoding Siglec-12 polypeptide. Analysis of human genomic contigs using the Celera human genome database identified the Siglec-12 sequence as being located on human chromosome 19q 13.4, approximately 1.2-1.3 megabases distal to Siglec-5.

### Example 5

### Tissues Expressing Siglec-12 Polypeptide

Oligonucleotide primers corresponding to predicted Siglec-12 polypeptide coding region sequences were used to assess Siglec-12 mRNA expression using a panel of human tissue and cell line cDNAs (“MTC cDNA,” Clontech). The forward primer: 5′-CAGCCTCTCCGTGCACTACCCTCCAC (SEQ ID NO:20) and reverse primer: 5′-GACCTCTTCACTTTGGAACCATCCCTGACATC (SEQ ID NO:21) were designed to amplify a predicted coding region fragment of approximately 750 bp in length corresponding to nucleotides 1395-2152 of SEQ ID NO:1. Since the forward primer crosses an intron, no genomic fragment would be predicted to be amplified using this primer pair. Tissues and cell lines that expressed Siglec-12 mRNA, as evidenced by the presence of an amplified DNA fragment of approximately 750 bp in length, included placenta, pancreas, ovary, liver, kidney, spleen, testis, stomach, esophagus, brain, heart, lung, colon, lymph node, bone marrow, fetal liver, fetal muscle, and fetal thymus. Negative tissues included skeletal muscle, thymus, prostate, small intestine, fetal brain, fetal lung, fetal spleen and fetal kidney. Because a siglec-like polypeptide of the invention is not expressed in every tissue, the invention provides a method of tissue-typing by utilizing antibodies to the polypeptides of the invention or by utilizing oligonucleotide primers or probes specific for polynucleotides of the invention.

### Example 6

### Binding Assay

Siglec-12 polypeptides or fragments thereof are expressed by recombinant DNA techniques, purified and tested for the ability to bind with various cells of the various lineages (e.g., hematopoietic cells). The binding assays employ Siglec-12 polypeptides, including soluble forms of these polypeptides, and oligomers formed as described below.

Oligomers for assays are prepared as follows. Fusion proteins comprising a leucine zipper peptide fused to the COOH-terminus of a Siglec-12 polypeptide are constructed as described above. Preferably, the polypeptide comprises a soluble form of Siglec-12 polypeptide, such as the extracellular region of a Siglec-12 polypeptide. An expression construct is prepared, essentially as described in Baum et al. (EMBO J. 13:3992-4001, 1994). The construct, in expression vector pDC409, encodes a leader sequence derived from human cytomegalovirus, followed by the leucine zipper moiety fused to the C-terminus of a soluble Siglec-12 polypeptide. Alternatively, a gene fusion encoding a Siglec-12 polypeptide/Fc fusion protein is inserted into an appropriate expression vector. Polypeptide/Fc fusion proteins are expressed in host cells transformed with the recombinant expression vector, and allowed to assemble by the formation of interchain disulfide bonds between the Fc moieties, thus yielding dimeric molecules.

The expressed Fc/Siglec-12 polypeptide or leucine zipper/Siglec-12 polypeptide fusion protein is contacted with a cell suspected of expressing a Siglec-12 polypeptide binding partner. In one embodiment, the activity of the fusion protein is measured by detecting a change in calcium mobilization in the cell expressing the cognate. In another embodiment, the activity of the fusion protein is measured by detecting the ability of a cell expressing a native Siglec-12 polypeptide to bind to or interact with a cell expressing a Siglec-12 polypeptide-binding partner in the presence and absence of the fusion protein. In yet another embodiment, the binding activity of the fusion construct is detected by detecting binding of the fusion protein to a Siglec-12 polypeptide cognate using, for example, a labeled anti-IgG antibody.

### Example 7

### Analysis of Siglec-12 Expression by Real-Time Quantitative PCR

RNA samples were obtained from a variety of tissue sources and from cells or tissues treated with a variety of compounds; these RNA samples included commercially available RNA (Ambion, Austin, Tex.; Clontech Laboratories, Palo Alto, Calif.; and Stratagene, La Jolla, Calif.). The RNA samples were DNase treated (part # 1906, Ambion, Austin, Tex.), and reverse transcribed into a population of cDNA molecules using TaqMan Reverse Transcription Reagents (part # N808-0234, Applied Biosystems, Foster City, Calif.) according to the manufacturer's instructions using random hexamers. Each population of cDNA molecules was placed into specific wells of a multi-well plate at either 5 ng or 20 ng per well and run in triplicate. Pooling was used when same tissue types and stimulation conditions were applied but collected from different donors. Negative control wells were included in each multi-well plate of samples.

Sets of probes and oligonucleotide primers complementary to mRNAs encoding Siglec-12 polypeptides were designed using Primer Express software (Applied Biosystems, Foster City, Calif.) and synthesized, and PCR conditions for these probe/primer sets were optimized to produce a steady and logarithmic increase in PCR product every thermal cycle between approximately cycle 20 and cycle 36. The forward primer used corresponded to nucleotides 1677 to 1696 of SEQ ID NO: 1 at a concentration of 900 nM; the reverse primer used corresponded to the complement of nucleotides 1729 to 1747 of SEQ ID NO:1 at a concentration of 300 nM. The FAM-labeled probe used for Siglec-12 corresponded to the complement of nucleotides 1699 to 1727 of SEQ ID NO:1 at a concentration of 200 nM. Oligonucleotide primer sets complementary to 18S RNA and to mRNAs encoding certain ‘housekeeper’ proteins—beta-actin, HPRT (hypoxanthine phosphoribosyltransferase), DHFR (dihydrofolate reductase), PKG (phosphoglycerate kinase), GUSB (beta-glucouronidase), and GAPDH (glyceraldehyde-3-phosphate dehydrogenase)—were synthesized and PCR conditions were optimized for these primer sets also. Multiplex TAQMAN PCR reactions using both Siglec-12 and GUSB probe/primer sets were set up in 25-microliter volumes with TAQMAN Universal PCR Master Mix (part # 4304437, Applied Biosystems, Foster City, Calif.) on an Applied Biosystems Prism 7700 Sequence Detection System. Threshold cycle values (C_{T}) were determined using Sequence Detector software version 1.7a (Applied Biosystems, Foster City, Calif.), and delta CT (the average FAM value minus the average VIC value) was calculated and transformed to 2E(−dC_{T}), which is 2 to the minus delta C_{T}, for relative expression comparison of Siglec-12 to GUSB.

Analysis of Siglec-12 expression relative to HPRT expression in a variety of adult and fetal RNA samples indicated that Siglec-12 is expressed more abundantly in adult heart, liver, ovary, spleen, fetal liver, and placenta.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A substantially purified polypeptide comprising a Siglec-12 polypeptide, wherein the amino acid sequence of the Siglec-12 polypeptide is at least 80% identical to a sequence as set forth in SEQ ID NO:2, wherein the Siglec-12 polypeptide binds a sialic acid moiety.

2. The substantially purified polypeptide of claim 1, wherein the amino acid sequence is at least 90% identical to a sequence as set forth in SEQ ID NO:2.

3. The substantially purified polypeptide of claim 1, wherein the amino acid sequence is a sequence as set forth in SEQ ID NO:2.

4. The substantially purified polypeptide of claim 1, wherein the sequence is from about amino acid 14 to 686 of SEQ ID NO:2.

5. The substantially purified polypeptide of claim 1, wherein the sequence is from about amino acid 14 to 549 of SEQ ID NO:2.

6. A substantially purified polypeptide comprising a Siglec-12 extracellular domain, wherein the amino acid sequence of the Siglec-12 extracellular domain is at least 80% identical to a sequence as set forth from about amino acid 14 to 549 of SEQ ID NO:2, wherein the Siglec-12 extracellular domain binds a sialic acid moiety.

7. A fusion polypeptide comprising a first polypeptide comprising an amino acid sequence as set forth from about amino acid 14 to 549 of SEQ ID NO:2 operably linked to a second polypeptide.

8. The fusion polypeptide of claim 7, wherein the second polypeptide is an Fc polypeptide.

9. The fusion polypeptide of claim 7, wherein the second polypeptide is a leucine zipper polypeptide.

10. The fusion polypeptide of claim 7, comprising a linker polypeptide separating the first polypeptide and the second polypeptide.

11. An isolated polynucleotide encoding a polypeptide of claim 1.

12. An isolated polynucleotide encoding a polypeptide of claim 6.

13. An isolated polynucleotide encoding a fusion polypeptide of claim 7.

14. An isolated polynucleotide comprising a sequence selected from the group consisting of:
a) SEQ ID NO:1;
b) SEQ ID NO:1 from about nucleotide 40 to 2058;
c) SEQ ID NO:1 from about nucleotide 40 to 1647;
d) sequences complementary to SEQ ID NO:1;
e) sequences complementary to SEQ ID NO:1 from nucleotide 40 to 2058;
f) sequences complementary to SEQ ID NO:1 from nucleotide 40 to 1647;
g) any of (a)-(f), wherein T can also be U; and
h) fragments of (a)-(g) that are at least 50 bases in length and that will hybridize under moderate to highly stringent conditions to a nucleic acid which encodes a polypeptide consisting of a sequence as set forth in SEQ ID NO:2.

15. A vector comprising a polynucleotide of claim 14.

16. The vector of claim 15, wherein the vector is a plasmid.

17. The vector of claim 15, wherein the vector is a viral vector.

18. A host cell containing the vector of claim 15.

19. A recombinant host cell comprising a polynucleotide of claim 14 under the control of a heterologous regulatory sequence.

20. The host cell of claim 19, wherein the cell is prokaryotic.

21. The host cell of claim 19, wherein the cell is eukaryotic.

22. A method of producing a polypeptide comprising culturing a host cell of claim 19 under condition that promote expression of the polypeptide.

23. A polypeptide produced by culturing a host cell of claim 19 under conditions that promote expression of the polypeptide.

24. A substantially purified antibody that specifically binds to a polypeptide consisting of a sequence as set forth in SEQ ID NO:2.

25. The substantially purified antibody of claim 24, wherein the antibody is a monoclonal antibody.

26. The substantially purified antibody of claim 24, wherein the antibody is a human or a humanized antibody.

27. A pharmaceutical composition comprising the antibody of claim 24.

28. A pharmaceutical composition comprising a polypeptide selected from the group consisting of:
(a) a polypeptide comprising a sequence as set forth in SEQ ID NO:2 from about amino acid 14 to 686; and
(b) a polypeptide comprising a sequence as set forth in SEQ ID NO:2 from about amino acid 14 to 549,
and a pharmaceutical carrier, excipient or diluent.

29. A method for identifying an agent which modulates expression of a polynucleotide comprising contacting a sample containing a polynucleotide comprising a sequence as set forth in SEQ ID NO:1 with a test agent and measuring the expression of the polynucleotide compared to a control, wherein a change in expression compared to the control is indicative of an agent that modulates expression of the polynucleotide.

30. The method of claim 29, wherein the agent is selected from the group consisting of a polypeptide, a peptide, a peptidomimetic, a nucleic acid, and a small molecule.

31. The method of claim 29, wherein the sample is a biological sample from a subject.

32. The method of claim 29, wherein the sample comprises cells.

33. The method of claim 29, wherein the change in expression is an increase in expression.

34. The method of claim 29, wherein the measuring is by PCR or Northern Blot.

35. The method of claim 29, wherein the measuring is by detecting a polypeptide expressed by the polynucleotide.

36. A method for identifying an agent which modulates the activity of a polypeptide comprising contacting a sample containing a polypeptide comprising a sequence selected from the group consisting of (a) SEQ ID NO:2, (b) SEQ ID NO:2 from 14 to 686, and (c) SEQ ID NO:2 from 14 to 549, with a test agent and measuring the activity of the polypeptide compared to a control, wherein a change in activity compared to the control is indicative of an agent that modulates activity of the polypeptide.

37. The method of claim 36, wherein the agent is selected from the group consisting of a polypeptide, a peptide, a peptidomimetic, a nucleic acid, and a small molecule.

38. The method of claim 36, wherein the sample is a biological sample from a subject.

39. The method of claim 36, wherein the sample comprises cells.

40. The method of claim 42, wherein the change in activity is an increase in activity.

41. The method of claim 36, wherein the measuring is by quantitating the amount of polypeptide in the sample.

42. A method of treating a siglec-associated disorder or disease comprising contacting a subject with a Siglec-12 polypeptide or Siglec-12 polynucleotide in an amount effective to treat the siglec-associated disorder or disease.

43. The method of claim 42, wherein the siglec-associated disorder is selected from the group consisting of a rheumatologic disorder, a bone marrow or solid organ transplant disorder, a graft-versus-host disorder, an inflammatory disorder, an autoimmune disorder, a neurologic disorder, a cell proliferative disorder, an infection, a cardiovascular disorder, a hematologic disorder, liver disorder, and a bone disorder.

44. The method of claim 42, wherein the Siglec-12 polypeptide has a sequence as set forth in SEQ ID NO:2 or a bioactive fragment thereof.

45. The method of claim 44, wherein the bioactive fragment has a sequence as set forth in SEQ ID NO:2 from about amino acid 14 to 549.

46. The method of claim 42, wherein the Siglec-12 polynucleotide has a sequence as set forth in SEQ ID NO:1.

47. The substantially purified antibody of claim 24 conjugated to a toxin or a radioisotope.

48. A method of treating a subject having a tumor that expresses a Siglec-12 polypeptide, comprising administering to the subject an antibody of claim 47.
